(19) United States
(12) Patent Application Publication
(10) Pub. No.: US 2021/0040485 A1
(43) Pub. Date: Feb. 11, 2021

(54) Elimination or Neutralization of Endogenous High Molecular Weight FGF-2 Increases Cardiac Resistance to Doxorubicin-Induced Damage

(71) Applicant: University of Manitoba, Winnipeg (CA)

(72) Inventors: Elissavet Kardami, Winnipeg (CA); Navid Koleini, Winnipeg (CA)

(21) Appl. No.: 16/977,614

(22) PCT Filed: Mar. 5, 2019
(86) PCT No.: PCT/CA2019/050262

§ 371 Date: Sep. 2, 2020

Related U.S. Application Data
(60) Provisional application No. 62/638,695, filed on Mar. 5, 2018.

(51) Int. Cl. C12N 15/113; C07K 16/22; A61P 39/00

(52) U.S. Cl. CPC C12N 15/1136; C07K 2317/76; A61P 39/00; C07K 16/22

(57) ABSTRACT

Doxorubicin (Dox), a potent anti-cancer drug, can cause cardiac dysfunction and failure. Endogenous Fibroblast Growth Factor-2 (FGF2), a protein implicated in cardioprotection, regulates cardiac vulnerability to Dox. In wild type mice and in humans, cardiac FGF2 is composed of a mixture of mostly high (Hi-) but also low (Lo) molecular weight isoforms and is produced mainly by non-myocytes. We compared wild type mice, FGF2(WT), to mice genetically engineered as to express: no FGF2, FGF2(−); only Hi-FGF2, FGF2(Hi); only Lo-FGF2, FGF2(Lo). Sole expression of endogenous L0-FGF2 in vivo, or by fibroblasts in vitro, protects cardiomyocytes from Dox. In a wild type environment, neutralization of endogenous Hi-FGF2 presents a potential prophylactic treatment against Dox-induced cardiotoxicity.

### PRIOR APPLICATION INFORMATION

The instant application claims the benefit of U.S. Provisional Patent Application Ser. No. 62/638,695, filed Mar. 5, 2018 and entitled “Elimination or neutralization of endogenous high molecular weight FGF-2 increases cardiac resistance to Doxorubicin-induced damage”, the entire contents of which are incorporated herein by reference for all purposes.

### BACKGROUND OF THE INVENTION

Doxorubicin (Dox)-induced cardiotoxicity is associated with development of heart failure in cancer patients and survivors (1). The incidence and severity of cardiotoxicity rise with increased cumulative doses of Dox during the cancer treatment (3-5% with 400 mg/m² to 18-48% with 700 mg/m²) (2). To date, the only FDA approved drug for prevention of Dox-induced cardiotoxicity is Dexrazoxane. Due to controversial results regarding the cancer-related outcomes (3, 4), Dexrazoxane use is limited (5). Investigating new strategies to minimize Dox-induced cardiac damage, which can manifest both acutely and chronically, stands to point to new cardioprotective strategies, and confer significant benefit to cancer patients.

Fibroblast Growth Factor 2 (FGF2), an endogenous heparin-binding growth factor produced predominantly by cardiac non-myocytes (fibroblasts), is reported to be cardioprotective in different models of injury including Dox-induced damage (6, 7). A recent study indicated that administered FGF2 can activate endogenous anti-oxidant and detoxification pathways during acute Dox treatment (8). It should be noted that the majority of studies on FGF2 cardioprotection rely on exogenous supplementation with, or over-expression of, the 18 kDa FGF2 isoform (6). Endogenous FGF2, however, is expressed as >20 kDa CUG initiated isoforms, collectively referred to as high molecular weight FGF2 (Hi-FGF2), which constitutes 70-80% of total expressed and ‘secreted’ FGF2, in addition to the AUG initiated 18 kDa isoform (the low molecular weight FGF2 (Lo-FGF2)) (6, 9, 10).

Fibroblast growth factor 2 (FGF2) is an endogenous heparin-binding multifunctional growth factor expressed and secreted predominantly by cardiac non-myocytes (fibroblasts) in the heart (6). Administered or overexpressed FGF2 is reported to be cardioprotective in different injury models including ischemia—as well as genotoxic drug-induced damage (6, 7, 35). Thus, it is reasonable to surmise that endogenous FGF2 would regulate cardiac vulnerability to stress stimuli; however, it should be noted that the majority of studies on FGF2 cardioprotection rely on exogenous supplementation with, or over-expression of, the 18 kDa FGF2 isoform (6). Activation of plasma membrane FGF2 tyrosine kinase receptor (s), FGFR1, downstream activities of protein kinase C (PKC), extracellular signal-regulated kinase (ERK) and protein kinase B (AKT) pathways and phosphorylation of the cellular and mitochondrial channel protein connexin-43 are implicated in the mechanism of FGF2 cardioprotection (7, 35, 42). In addition, a recent study showed that administered FGF2 is protective by activating endogenous anti-oxidant and detoxification pathways (Nuclear factor (erythroid-derived 2)-like 2 (Nrf2)/hemoxygenase-1(HO-1)) during acute Doxorubicin (Dox) treatment (8). Endogenous FGF2 is expressed as >20 kiloDalton (kDa) leucine (CUG)-initiated isoforms, collectively referred to as high molecular weight FGF2 (Hi-FGF2) and constituting 70-80% of total expressed and ‘secreted’ FGF2, in addition to the methionine (AUG)-initiated low molecular weight FGF2 (18 kDa; Lo-FGF2) (6, 9, 10). The role of endogenous FGF2 in the context of cardiac response to genotoxic agents such as Dox in vivo is not known. Here, we provide evidence that wild type mice, expressing both Hi and Lo-FGF-2 isoforms, are more vulnerable to Dox-induced cardiac damage when compared to mice expressing only Lo-FGF2. Furthermore, elimination or neutralization of endogenous Hi-FGF2 reduces cardiac vulnerability to Dox treatment.

Specifically, FGF-2 isoforms arise from the same mRNA transcribed from the FGF-2 gene. The human mRNA constitutes 5 different initiation codons (1 AUG and 4 CUGs) for protein synthesis. As a result, 5 different proteins which they are different in size can be made from a single mRNA. The smallest isoforms (18 KDa (154 amino acids)) is called Lo-FGF-2 which is synthesized from AUG initiation site. All larger isoforms (CUG initiated) are collectively called Hi-FGF-2 (22 kDa (199 amino acids); 22.5 kDa (204 amino acids); 24 kDa (213 amino acids); and 34 KDa (291 amino acids)).

The role of endogenous FGF2 in the context of cardiac response to Dox in vivo is not known.

### SUMMARY OF THE INVENTION

According to an aspect of the invention, there is provided a method of reducing severity of cardiotoxicity caused by administration of an anthracycline to an individual comprising administering to said individual an effective amount of an FGF-2 modulating compound.

According to another aspect of the invention, there is provided a method of reducing severity of cardiotoxicity in an individual who had been administered an anthracycline comprising administering to said individual an effective amount of a FGF-2 modulating compound on a schedule.

### BRIEF DESCRIPTION OF THE DRAWINGS

FIG. 1. The effect of FGF2 expression and composition on Fractional Shortening (FS) post-Dox, in (A) male, and, (B), female mice (A): Sample size: 9-10 for FGF2(WT), 3-10 for FGF2(−), 8-10 for FGF2(Hi), and 9-10 for FGF2(Lo). (B): Sample size, n=10 for all groups. For each time point, the Dox-treated groups (Red) was compared to the saline-treated group; asterisk points to significant differences (P<0.05). In A and B, compare the panels surrounded by dashed lines. Dox-treatment causes decreased function in FGF2(WT) but not FGF2(Lo).

FIG. 2. The effect of FGF2 expression and composition on Ejection Fraction (EF) post-Dox, in (A) male, and, (B), female mice. (A): Sample size: 9-10 for FGF2(WT), 3-10 for FGF2(−), 8-10 for FGF2(Hi), and 9-10 for FGF2(Lo). (B): Sample size, n=10 for all groups. For each time point, the Dox-treated groups (Red) was compared to the saline-treated group; asterisk points to significant differences (P<0.05). In A, and B, compare the panels surrounded by dashed lines. Dox-treatment causes decreased function in FGF2(WI) but not FGF2(Lo).

FIG. 3. The effect of FGF2 expression and composition on Left Ventricular Diastolic Diameter (LVEDD) post-Dox, in (A) male and (B) female mice (A): Sample size: 9-10 for FGF2(WT), 3-10 for FGF2(−), 8-10 for FGF2(Hi), and 9-10 for FGF2(Lo). (B): Sample size, n=10 for all groups. For each time point, the Dox-treated groups (Red) were compared to the saline-treated group; asterisk points to significant differences (P<0.05). In A, compare the panels surrounded by dashed lines. Dox-treatment causes decreased increased damage in FGF2(WT) but not FGF2(Lo).

FIG. 4. The effect of FGF2 expression and composition on relative Bnip3 protein accumulation post-Dox, in (A) male and (B) female mice. Graphs show cumulative densitometry measurements for Bnip3 (western blots), corrected for loading variations using corresponding total lane staining for Ponceau S. Inserts show representative western blot bands. Comparisons are made between saline (green) and Dox-treated (red) groups for each mouse strain. Brackets point to statistically significant differences (P<0.05).

FIG. 5. Cardiomyocytes were co-cultured with mouse embryonic fibroblasts (MEFs) of different FGF-2 transgenic animals for 24 hours and then insulted with Dox for 24 hours. (A) shows Troponin T release in the media of co-cultures 24 hours after Dox insult. Cardiomyocytes co-cultured with FGF2 (Lo) MEFs were protected against Dox damage, while the ones co-cultured with FGF2 (WT) MEFs showed more vulnerability against Dox. Two-way ANOVA (p<0.05) was used to analyze the data. (B) Mitochondrial permeability transition pore formation was assessed using Calcein-Cobalt assay. One-way ANOVA was used to compare the groups.

FIG. 6. Cardiomyocytes were co-cultured with human fibroblast-FibiPSC (A), co-cultured using transwells (B), or cultured in the conditioned media of FibiPSC (C) in the presence Hi-Ab or rabbit IgG (20 ug/ml). Troponin release was measured by western blotting (A and B) or LDH release assay (C). (D) Cardiomyocytes were cultured in the conditioned media of FibiPSC in the presence of total FGF-2 neutralizing antibody or mouse IgG. Dox-induced LDH release was measured using LDH activity kit.

FIG. 7. Heparin-sepharose-bound FGF2 from 200 ug of cardiac extract, detected using anti-(total)-FGF2 antibodies. As expected, no signal is present in FGF2(−) extracts, and: FGF2(Hi) extracts contain only Hi-FGF2, FGF2(Lo) extracts contain only Lo-FGF2, while FGF2(WT) extracts contain predominantly Hi-but also Lo-FGF2.

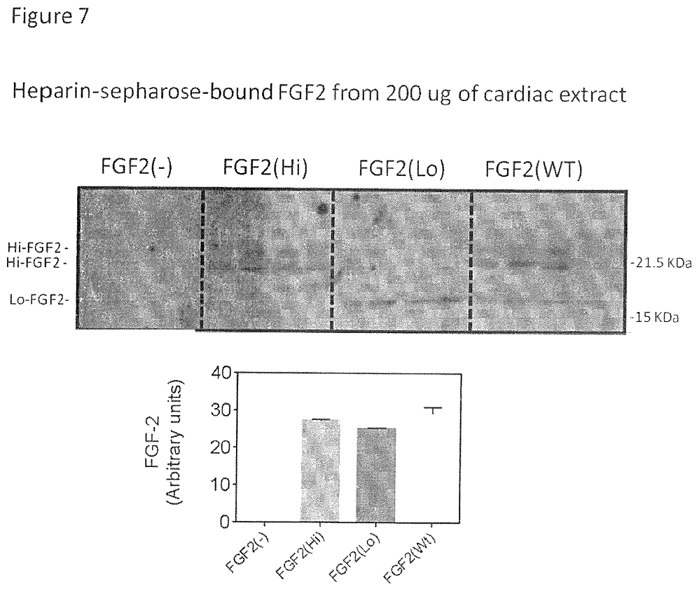

FIG. 8. The effect of FGF2 expression and composition on mouse body weight at (A) baseline, and (B) in saline (green column) or Doxorubicin (Red column)-injected mice, at 10 days post-injection. Doxorubicin caused significantly decreased body weight in all groups (P<0.05). Body weight values in groups indicated by an asterisks (*) in (A) are significantly different than all other groups.

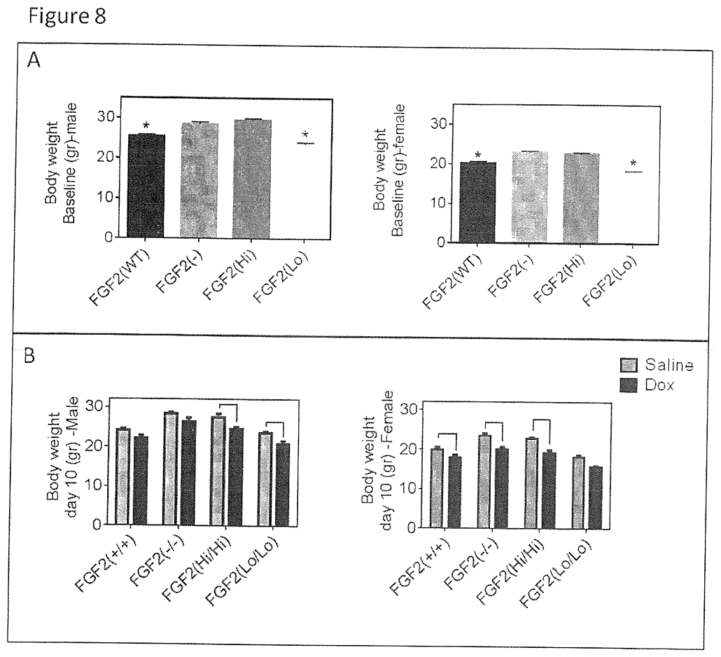

FIG. 9. Kaplan-Meier graphs shows the mortality in the male (A) and female (B) transgenic mice of Dox and Saline treated groups. Dox treated FGF2(−) male mice showed a significant increase in mortality compared to Saline ones. The trend has been observed in the female FGF2(−) mice, although it was not statistically significant.

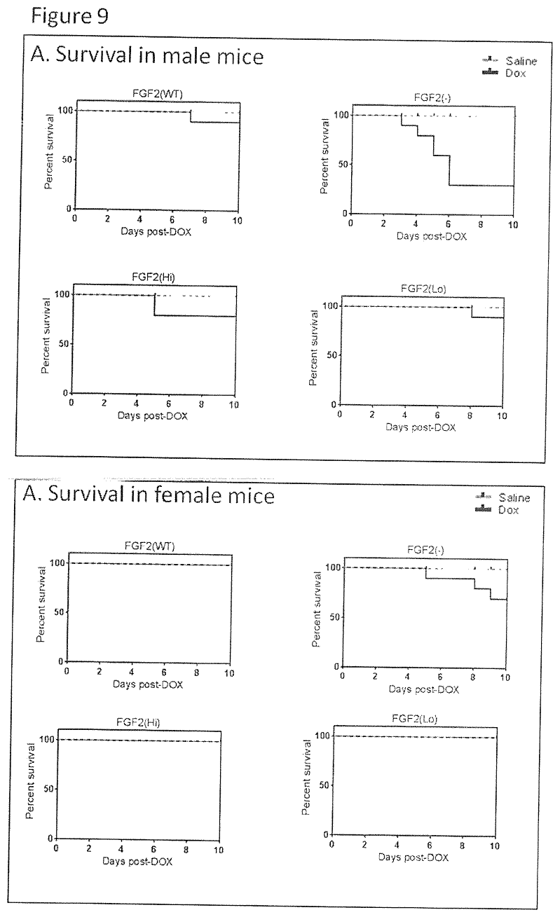

FIG. 10. Western blot analysis of FGF2 composition, using an antibody recognizing all FGF2 isoforms. Lane 1, extracts from human FibiPSC are seen to contain Lo-FGF2 (18 kDa) and Hi-FGF2 (>20 kDa). Lane 2 shows that material immunoprecipitated from human FibiPSC with Hi-Ab contains only Hi-FGF2. Lane 3 shows FGF2 remaining in human FibiPSC extracts after immunoprecipitation with Hi-Ab.

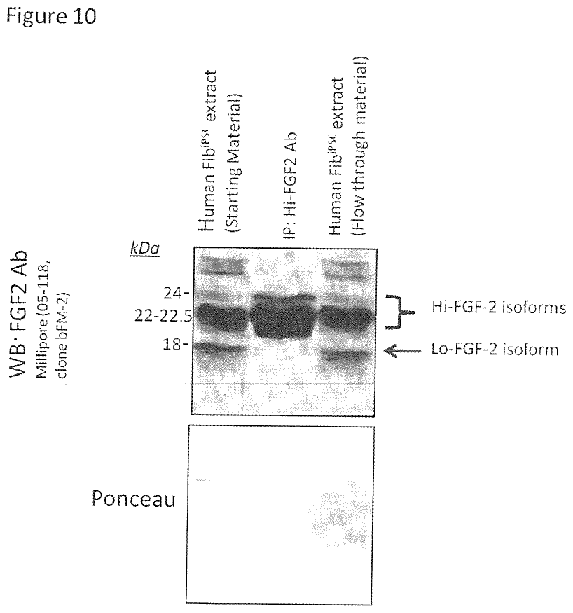

FIG. 11. MCF-7 (breast cancer) cells were incubated with rabbit antibody (IgG, 20 ug/ml) or Hi-Ab (20 ug/ml) for 24 hours. After 24 hours, the media was supplemented again with IgG or Hi-Ab. Dox (0.5 uM) was added to the cells and LDH activity in the media of cells were measured using Pierce™ LDH Cytotoxicity Assay Kit (Thermofisher #88953) according to the manufacturer protocols. This figure shows that the Hi-Ab treatment does not affect the ability of Dox to kill at least some kind of cancer cells.

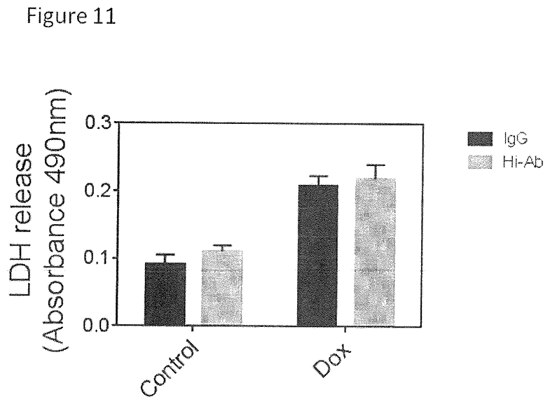

FIG. 12. The effect of endogenous Hi-FGF2 elimination on Ejection Fraction (% EF) post-Doxorubicin injection, in (A) Male (A) and (B) Female wild type (FGF2(WT)) or Hi-FGF2-depleted (FGF2(Lo)) mice, as indicated. Panels (i) and (ii) show % EF at baseline and up to 10 days post Dox in sham, saline-injected (green squares) versus Dox-injected (red circles) animals, in FGF2(WT) and FGF2(lo) groups, respectively. Asterisks point to significant differences between the Dox-treated group and its corresponding sham group at the same time point (p<0.05). Panel (iii) shows EF of the FGF2(WT), purple triangle, and the FGF2(Lo), blue circle, groups before Dox (baseline), and at 1 day and 10 days post-Dox. Brackets mark groups with statistically significant differences from each other. Sample sizes in (A) are n=9-10 in Dox and n=10 in saline treated groups for both FGF2(WT) and FGF2(Lo), and in B, n=5 and 6 for the FGF2(WT) saline and Dox groups, respectively; n=10 for the FGF2(Lo) groups. Two-way ANOVA and Sidak post hoc tests were used to analyze the data.

FIG. 13. Elimination of endogenous Hi-FGF2 prevents the Doxorubicin-induced increase in Left Ventricular End Diastolic Dimension (LVEDD). Panels A and B show LVEDD values at baseline, and at 1 and 10 days post-Dox in FGF2(WT) (purple triangles) and FGF2(Lo) (blue circles) in male and female mouse groups, respectively. Values for the FGF2(WT) groups are compared to those of the FGF2(Lo) group at each time point; comparisons were also made within each group, comparing values at baseline to those at 10 days post-Dox. Significant differences between groups are marked by brackets, p<0.05. In (A), sample size is n=9-10. In (B) sample size is n=10. and 6 for FGF2(WT) saline and Dox groups, respectively. and n=10 for FGF2(Lo) groups. Two-way ANOVA and Sidak post-hoc tests were used to analyze the data. Asterisk points to significant differences between the Dox-treated group and the saline-treated group at the same time point (P<0.05).

FIG. 14. Elimination of Hi-FGF2 prevents Bnip3 upregulation in male mice. Western blot-based analyses of the anti-Bnip3 signal in (A) male, and (B), female FGF2(WT) or FGF2(Lo) groups, as indicated, at 10 days after Dox- or saline-injection. The measurement from the lane marked with an asterisk was excluded because it was a significant outlier. Densitometry values from each saline-injected group were arbitrarily assigned a value of 1, and values from the Dox-injected groups were adjusted accordingly. Sample size n=4 for all groups except male FGF2(Lo) where n=3. Brackets denote significant differences between groups, at P<0.05, Student's t-test. Please note that four different western blots were used (and shown) to allow simultaneous analysis of the Bnip3 signal between the Dox-treated and its corresponding saline group.

FIG. 15. Elimination of Hi-FGF2 from mouse embryonic fibroblasts MEFs) protects cardiomyocytes (in co-culture) from Dox-induced injury and death. Panel A shows a western blot of supernatants obtained from cardiomyocyte-MEF co-cultures and probed for cTnT; Ponceau S staining of the same membrane is also shown. Myocytes were co-cultured with FGF2(WT) or FGF2(lo)-derived MEFs and exposed (red squares) or not (green circles) to Dox, as indicated. All lanes shown were obtained from the same gel. Panel B shows quantitation of the cTnT signal, adjusted by the Ponceau staining, from Panel A, n=4. Brackets show significant differences (P<0.05) between groups. Two-way ANOVA (Fisher's LSD post hoc) was used to analyze the data. In Panel C shows relative calcein fluorescence intensity of cells in co-cultures, stained using the Calcein-Cobalt assay to visualize mitochondrial permeability pore formation, seen as a loss of ‘green’ signal. Fluorescence was measured using Image J (n=160 in myocyte-MEF-FGF2(WT), orange circles), and n=168 in myocyte-MEF-FGF2(Lo) co-cultures (blue circles), as indicated. A Student's t-test was used to compare the groups, and brackets denote significant differences, P<0.05. Panel D shows representative fluorescence images of the co-culture groups, stained for calcein (green) and counterstained with Mito-tracker (Red), to visualize mitochondria. Panels A1, 2, 3 show the same field from myocyte-MEF-FGF2(WT) co-cultures stained for Calcein, Mitotracker, as well a merge of both stains. Panels B1, 2, 3 show the same field from myocyte-MEF-FGF2(Lo) co-cultures stained for Calcein, Mitotracker, as well a merge of both stains. Purple sizing bar in A1 corresponds to 20 um.

FIG. 16. Antibodies to human high-molecular-weight fibroblast growth factor 2 (Hi-FGF2) (Hi-Ab) interact selectively with 22- to 24-kDa Hi-FGF-2. Shown are immunoblot analyses of FGF-2 isoforms present in h-Fib^{iPSC} cell extracts before and after immunoprecipitation (IP) with Hi-Ab (rabbit polyclonal) using a mouse monoclonal antibody recognizing all FGF2 isoforms. WB, Western blot. In lane 1, extracts from h-Fib^{iPSC} contain low molecular-weight FGF-2 (Lo-FGF2) (18 kDa) and Hi-FGF2 (>20 kDa). Lane 2 contains anti-FGF2 immunoreactive proteins remaining in the extract after removal of the Hi-Ab-immunoprecipitated material. Lane 3 contains material immunoprecipitated with Hi-Ab from a total of 150 μg protein extracted from Fib^{iPSC} and presenting a signal for only 22- to 24-kDa Hi-FGF2. Lane 1 was obtained from a different immunoblot and shows the presence of Hi- and Lo-FGF2 as concentrated from 40 ml of h-Fib^{iPSC}-conditioned medium using heparin-Sepharose beads. Ponceau red staining of the corresponding immunoblots is included.

FIG. 17. Neutralization of paracrine-acting human fibroblasts (FibiPSC)-produced high-molecular-weight fibroblast growth factor 2 (Hi-FGF2) attenuates doxorubicin (Dox)-induced cardiomyocyte damage. A: protein immunoblot detection and quantification of the cardiac troponin T (cTnT) signal in the supernatant from cocultures of cardiomyocytes with human FibiPSC, exposed or not to Dox, in the presence of control IgG or anti-Hi-FGF2 antibodies (Hi-Ab), as indicated. The corresponding Ponceau S staining is also shown. Cumulative data from densitometry of the two Dox-treated groups, adjusted for Ponceau staining, are shown in the included graph. The bracket denotes statistically significant differences between the two groups, n=3. A two-tailed Student's t-test was used to analyze data, P<0.05. B: immunoblot detection and quantification of the cTnT signal in the supernatant of cardiomyocytes exposed to secreted materials from human FibiPSC grown in inserts in a Transwell setup. Corresponding Ponceau S staining is also shown. Myocytes were insulted, or not, with Dox in the presence of Hi-Ab or control IgG, as indicated. Cumulative data from densitometry of the two Dox-treated groups, adjusted for Ponceau S staining, are shown in the included graph. The bracket denotes statistically significant differences between the two groups, n=3. A two-tailed Student's t-test was used to analyze data, P<0.05. C: lactate dehydrogenase (LDH) in the supernatant of cardiomyocytes exposed to human FibiPSC-conditioned medium in the presence of control IgG or Hi-Ab and treated or not with Dox, as indicated (n=3). Brackets show significant differences between the Dox-treated groups; Hi-Ab reduced Dox-induced damage. D: LDH in the supernatant of cardiomyocytes exposed to human FibiPSC-conditioned medium in the presence of control IgG or neutralizing antibodies (Neu-Ab) [neutralizing both Hi-FGF2 and low-molecular-weight FGF2 (Lo-FGF2)] and treated or not with Dox, as indicated (n=3). Brackets show significant differences between the Dox-treated groups; Neu-Ab increased Dox-induced damage. Two-way ANOVA (Sidak post hoc) was used to analyze data in C and D.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference for all purposes.

Doxorubicin (Dox), a potent anti-cancer drug, can cause cardiac dysfunction and failure. Doxorubicin IS a non-selective class I anthracycline antibiotic and a potent chemotherapeutic agent which is used for the treatment of numerous cancers.

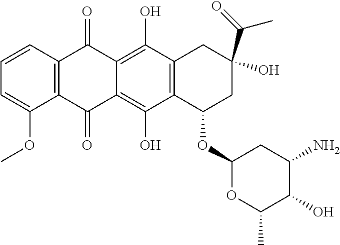 [SMILES: COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1]

Anthracyclines are used to treat various cancers. Doxorubicin and its derivative epirubicin are used in breast cancer, gastroesophageal cancers, childhood solid tumors, soft tissue sarcomas, and aggressive lymphomas aggressive lymphomas. The first drug of this class was daunorubicin. Cardiotoxicity is the common side effects of all these drugs.

As discussed below, Endogenous Fibroblast Growth Factor-2 (FGF2), a protein implicated in cardioprotection, regulates cardiac vulnerability to Dox. In wild type mice and in humans, cardiac FGF2 is composed of a mixture of mostly high (Hi-) but also low (Lo-) molecular weight isoforms and is produced mainly by non-myocytes. To investigate the role of endogenous FGF2 on cardiac vulnerability to Dox in vivo, we compared wild type mice, FGF2(WT), to mice genetically engineered as to express: no FGF2, FGF2(−); only Hi-FGF2, FGF2(Hi); and only Lo-FGF2, FGF2(Lo).

As discussed below, to address the role of non-myocyte-produced FGF2 we used co-cultures of neonatal rat cardiomyocytes with mouse embryonic fibroblasts (MEFs) from the different FGF2 strains. Cardiomyocytes co-cultured with FGF2 (Lo)-MEFs were resistant to Dox-induced injury. Cardiomyocytes co-cultured with human fibroblasts, or exposed to human fibroblast conditioned medium, developed increased resistance to Dox-induced injury in the presence of neutralizing anti-human Hi-FGF2.

Surprising, as discussed below, sole expression of endogenous Lo-FGF2 in vivo, or by fibroblasts in vitro, protects cardiomyocytes from Dox. In a wild type environment, neutralization of endogenous Hi-FGF2 presents a prophylactic treatment against Dox-induced cardiotoxicity. In the work presented here we provide evidence that: FGF2 expression in the heart of wild type mice is not protective from Dox-induced toxicity compared to the expression of only Lo-FGF2; and removal or neutralization of endogenous Hi-FGF2, but not Lo-FGF2, reduces cardiac vulnerability to Dox.

As discussed herein, we have examined how endogenous expression of FGF2 isoforms, individually and together, as well as complete lack of endogenous FGF2, may influence cardiac vulnerability to Dox. Major novel findings presented here are: (1) exclusive Lo-FGF2 expression, either in vivo, or by fibroblasts in co-cultures, promoted cardiac resistance to Dox-induced loss of cardiac function and myocyte injury; (2) complete lack of FGF2 resulted in high mortality and overall worse cardiac function post-Dox; (3) the wild type FGF2 phenotype (simultaneous expression of Hi- and Lo-FGF2 isoforms in hearts in vivo) presented reduced resistance to Dox-induced cardiac effects compared to the expression of either isoform on its own; (4) in a wild type background, blocking Hi-FGF2, but not Lo-FGF2, with a specific antibody protects cardiomyocytes from Dox-induced injury. Most of our findings were not sex dependent, with some exceptions: in the complete absence of endogenous FGF2, female sex decreased overall mortality and protected from the increased Left Ventricular End Diastolic Diameter (LVEDD) post-Dox observed in males; the pattern of Bnip3 upregulation in response to Dox in the various FGF2 groups, was different between male and female groups.

Amongst males, the inability of Dox to upregulate the pro-cell death Bnip3 protein was associated with the cardioprotected phenotype of the FGF2(Lo) group.

According to an aspect of the invention, there is provided a method of reducing severity of cardiotoxicity caused by administration of an anthracycline to an individual comprising administering to said individual an effective amount of an FGF-2 modulating compound.

Accordingly, administration of an effective amount of an FGF-2 modulating compound will accomplish at least one of the following: reducing severity of damage to cardiomyocytes; reducing the decrease in EF; and reducing the increase in LVEDD compared the heart of an individual of similar age and condition administered an approximately equivalent amount of an anthracycline without coadministration of an FGF-2 modulating agent. In some embodiments, the FGF-2 modulating agent is administered to the individual at least 3 hours before anthracycline administration, 1-3 hours before anthracycline administration, within 3 hours of anthracycline administration or shortly after anthracycline administration.

In some embodiments, said FGF-2 modulating compound increases Lo-FGF-2 isoform levels relative to Hi-FGF-2 isoform levels.

For example, excess Lo-FGF2 in above-physiological concentrations may be administered to the individual, either locally or systemically.

In some embodiments, said FGF-2 modulating compound decreases Hi-FGF-2 isoform levels relative to Lo-FGF-2 isoform levels.

For example, such an FGF-2 modulating compound may be (1) an antibody specific for Hi-FGF2. As discussed herein, the sequences of the FGF-2 isoforms are well-known and as such one of skill in the art could easily generate antibodies specific for Hi-FGF-2 in addition to those antibodies which are commercially available; (2) a non-coding RNA-based modulator, for example, a micro-RNA, or si-RNA, designed to selectively reduce Hi-FGF2 levels; (3) an anti-sense oligonucleotide-based modulator. Antisense oligonucleotides are single strand DNA sequences (around 20 nt) which can alter RNA translation. Upon dimerizing with the complementary sequence of the mRNA they physically hinder movement of ribosomes thus prevent protein synthesis. As FGF-2 mRNA has 4 CUG initiation and 1 AUG initiation codon, designing an ASO at 5′ end of the mRNA (distal to the AUG) can potentially lead to blockage of Hi-FGF-2 isoforms production and only synthesis of Lo-FGF-2; (4) a proteolytic-enzyme-based modulator which truncates Hi-FGF2 proteins so as to convert these proteins to a version functionally similar to Lo-FGF2.

In some embodiments, the anthracycline is doxorubicin, epirubicin or daunorubicin.

In other embodiments, the anthracycline is doxorubicin or epirubicin.

In some embodiments, the individual is undergoing chemotherapy for cancer.

In some embodiments, the FGF-2 modulator is administered prior to, or simultaneously with or shortly after anthracycline administration.

In some embodiments, the FGF-2 modulator is a Hi-FGF-2-specific antibody.

As discussed herein, the sequence for which such an antibody is made for is a sequence that is common in all the Hi-FGF-2 isoforms so that the antibody can bind and immobilize the 22, 22.5, and the 24 KDa isoforms. The 34 KDa band is not secreted and usually not seen in physiologic conditions. The Hi-FGF2 forms contain an additional 45-137 amino-acids compared to Lo-FGF2.

In some embodiments, the antibody binds to a 12-mer which is present only in the unique N-terminal extension of all Hi-FGF isoforms, as discussed below.

In some embodiments, the FGF-2 modulating compound, for example, a Hi-FGF-2 specific-Ab should be administered 1-3 hours before Dox administration. This time will give the antibody enough time to bind and inhibit circulating and also extracellular matrix bound FGF-2.

Anthracyclines are usually administered in divided doses over a period of time ranging from every other day, weekly, to every 10 days. In addition, based on the remission state of the cancer their administration may start again after a few months.

According to an aspect of the invention, there is provided a method of reducing severity of cardiotoxicity caused by administration of an anthracycline comprising administering to an individual who has been or who will be administered anthracycline an effective amount of an FGF-2 modulating compound.

According to an aspect of the invention, there is provided a method of reducing severity of cardiotoxicity caused by administration of an anthracycline comprising administering to an individual who has been or who will be administered anthracycline an effective amount of an FGF-2 modulating compound.

According to an aspect of the invention, there is provided a method of reducing severity of cardiotoxicity caused by administration of an anthracycline comprising reducing severity of damage to cardiomyocytes or reducing the decrease in EF or 1 educing the increase in LVEDD in the heart of an individual who has been or who will be administered anthracycline by administering an effective amount of an FGF-2 modulating compound to said individual, compared to a individual of similar age and condition administered an approximately equivalent amount of an anthracycline without coadministration of an FGF-2 modulating agent.

Use of the FGF-2 modulating compound will accomplish at least one of the following: reducing severity of damage to cardiomyocytes; reducing the decrease in EF; and reducing the increase in LVEDD in the heart of an individual who has or who will be administered anthracycline compared to the heart of a second individual of similar age and condition administered an approximately equivalent amount of an anthracycline without coadministration of an FGF-2 modulating agent. In some embodiments, the FGF-2 modulating agent is administered to the individual at least 3 hours before anthracycline administration, 1-3 hours before anthracycline administration, within 3 hours of anthracycline administration or shortly after anthracycline administration.

According to another aspect of the invention, there is provided an FGF-2 modulating compound for reducing severity of cardiotoxicity caused by anthracycline administration.

According to another aspect of the invention, there is provided an FGF-2 modulating compound for reducing severity of damage to cardiomyocytes from anthracycline in an individual administered anthracycline.

According to another aspect of the invention, there is provided an FGF-2 modulating compound for reducing severity of damage to cardiomyocytes from anthracycline in an individual administered anthracycline compared to cardiomyocytes of a second individual administered an approximately equivalent amount of an anthracycline without coadministration of an FGF-2 modulating agent.

According to another aspect of the invention, there is provided an FGF-2 modulating compound for reducing the decrease in EF in the heart of an individual administered anthracycline.

According to another aspect of the invention, there is provided an FGF-2 modulating compound for reducing the decrease in EF in the heart of an individual administered anthracycline compared to a second or control individual preferably of similar age and condition administered an approximately equivalent amount of an anthracycline without coadministration of an FGF-2 modulating agent.

According to another aspect of the invention, there is provided an FGF-2 modulating compound for reducing the increase in LVEDD in the heart of an individual administered anthracycline.

According to another aspect of the invention, there is provided an FGF-2 modulating compound for reducing the increase in LVEDD in the heart of an individual administered anthracycline compared to the heart of a second or control individual preferably of similar age and condition administered an approximately equivalent amount of an anthracycline without coadministration of an FGF-2 modulating agent.

In these embodiments, the FGF-2 modulating compound may be selected from the group consisting of:

(1) an antibody specific for Hi-FGF2; (2) a non-coding RNA-based modulator designed to selectively reduce Hi-FGF2 levels; and (3) an anti-sense oligonucleotide-based modulator.

As will be appreciated by one of skill in the art, repeat administrations of the antibody are likely necessary and depend on the circulating life time of the antibody in the blood stream. However, while not wishing to be bound to a particular theory or hypothesis, it is anticipated that a repeat of the antibody is required before every Dox administration and then, after discontinuation of the Dox therapy, every 10 days to 1 month until cardiac function is monitored to be normal for 6 months.

While not wanting to be bound to a particular theory or hypothesis, we anticipate that one injection of the Hi-Ab will result in appearance of the Hi-Ab in the blood which can last for 10 days to 1 month. We anticipate Hi-AB to present a similar life-span within the range of other antibody-based reagents used in the clinic. For example, the half life of infliximab (monoclonal antibody against TNF) is 9.5 days, Trastuzumab (monoclonal antibody against HER-2) is 5.8 days, and for pertuzumab it is 18 days. In another aspect of the invention, there is provided a method of reducing severity of cardiotoxicity in an individual who had been administered an anthracycline comprising administering to said individual an effective amount of an FGF-2 modulating compound on a schedule.

In some embodiments, the schedule is every 10 days to 1 month until cardiac function is monitored to be normal for 6 months.

As will be appreciated, the individual in this embodiment is an individual who was being administered an anthracycline as discussed above for example as a therapeutic for cancer, but the administration of anthracycline has ceased. As such, the individual is an individual who was receiving anthracycline but is no longer receiving anthracycline.

FGF2 Expression and Cardiac Vulnerability to Dox

In assessing how FGF2 expression and composition in the various mouse groups related to the Dox-induced cardiac effects, it was evident that the very early, up to 4 days post-Dox, responses were distinct from those at later time points, such as 5-10 days post-Dox. Within the very early time frame, it was unexpected to find that only FGF2(WT), but not FGF2(−), FGF2(Hi) or FGF2(Lo) mice, manifested detectable, if marginal, decreases in Cardiac Ejection Function (EF), Cardiac Fractional Shortening (FS), and increase in LVEDD. These findings indicate that the physiological expression and composition of FGF2 may sensitize mouse hearts, while, paradoxically, complete absence of FGF2, exerts an apparent protection from the very early cardiac effects of acute Dox. Our data of very early Dox-injury in wild type mice are in agreement with a previous study using a similar model of acute Dox toxicity (16), wherein Feridooni et al detected myocardial damage, measured as loss in cytoskeletal proteins, at 3 days post-Dox. As inflammation plays an important role in Dox-cardiotoxicity, while not wishing to be bound to a particular theory or hypothesis, one can speculate that, in the wild type mice, the combined activity of FGF2 isoforms in the heart and also systemically, promotes an early unfavorable inflammatory response, which could be attenuated in the other groups. There is currently no information as to how endogenous FGF2 isoform expression and composition affects local and systemic inflammation after Dox, or indeed after any kind of injury. It should be noted that the small decrease in function observed very early in the FGF2(WT) groups would not be considered clinically important.

The main thrust of the in vivo work is represented by findings at the 5-10 day post-Dox time frame. By day 10, and taking into consideration all echocardiography measurements, the complete lack of FGF2 seemed to result in maximally detrimental effects, such as a dramatic drop in FS, and substantially increased LVEDD at least amongst the male groups. By comparison, FGF2(WT), despite early evidence of vulnerability, displayed, by day 10, more modest decline in FS, or increase in LVEDD, compared to the FGF2(−) group. The LVEDD data are in agreement with a previous report that FGF2 knockout mice are predisposed to develop a pronounced cardiac dilatation in response to a stress stimulus such as hypertension, or myocardial infarction, compared to wild type mice (17) (18). Endogenous FGF2 expression is required for repair after injury due to myocardial infarction; absence of FGF2 in a knockout model reduced non-myocyte proliferation, collagen deposition and scar contraction after infarction, ultimately causing dilatation and compromising cardiac function (18). In a similar manner, it is possible that FGF2 expression allows some degree of heart repair to take place in the FGF2(WT) mice, thus reducing the magnitude of LVEDD changes observed compared to the FGF(−) mice. There is evidence for a reparative process taking place after 3 days post-Dox in wild type mice (19).

Major novel findings presented here are: (1) exclusive endogenous Lo-FGF2 expression, either chronically in vivo or only by fibroblasts in short-term co-cultures, raised cardiac resistance to Dox-induced loss of cardiac function or cardiomyocyte injury, respectively; (2) the wild type FGF2 phenotype, which consists of expression of both Hi- and Lo-FGF2 isoforms in a 7:3 ratio in hearts in vivo, is more vulnerable to Dox-induced deleterious effects compared to the exclusive expression of Lo-FGF2; (3) in a wild type genetic background, blocking non-myocyte Hi-FGF2 but not Lo-FGF2 with a specific antibody protected cardiomyocytes from Dox-induced injury.

Endogenous Lo-FGF2 Expression Increases Cardiac Resistance to Dox

Our studies demonstrated that the FGF2(Lo) mice, male and female, were clearly protected from Dox-induced deleterious effects on cardiac systolic function and LVEDD. This raises the question of whether the protective effect of Lo-FGF2 is suppressed in FGF2(WT) mice, possibly by the concomitant expression of Hi-FGF2. However, mice exclusively expressing Hi-FGF2 appeared protected from Dox up until day 7, which would argue that, in a background of complete absence of Lo-FGF2, Hi-FGF2 is in fact cardioprotective, at least up to a certain time-point post-Dox. By day 10, FGF2(Hi) mice did manifest decreases in systolic function, and fared worse than the FGF2(Lo) groups. Our findings from the FGF2(Hi) mice are in general agreement with our previous studies showing that: (a) Exogenous recombinant Hi-FGF2 protected cardiomyocytes from Dox-induced injury, in vitro, measured at 24 h post-Dox (8); (b) Hi-FGF2 intracardiac injection exerted acute, but not sustained, protection from myocardial infarction-induced loss of contractile function (20-23).

Although the hearts of adult female animals are generally considered to be more resistant to injurious stimuli compared to males, no sex-related differences were identified regarding vulnerability to Dox and FGF2 isoform expression, at least within the ‘acute’ time frame of our studies and based on the parameters measured.

Dox is a known mitochondrial toxin forming mitochondrial permeability transition pores leading to apoptosis and necrosis (26, 27). There is evidence that Bnip3, which is localized to cardiac mitochondria via its transmembrane domain, is central in Dox-induced mitochondrial perturbation and eventual cell death (28). Several in vivo studies have reported that Dox upregulated cardiac Bnip3 in male mice. In agreement, in our male groups, Dox upregulated Bnip3 protein only in the FGF2(WT), while no changes were seen in the FGF2(Lo) group. These findings suggested that Dox-induced cardiotoxicity may be mitigated in FGF2(Lo) mice by prevention of Bnip3 upregulation. Our recent in vitro studies support this notion: pre-exposure of cardiomyocytes to Lo-FGF2 prevented the Dox-induced upregulation of p53 as well as its downstream target Bnip3, by an mTOR/Nrf2/HO-1 dependent pathway (8).

As selective elimination of Hi-FGF2 isoforms in the FGF2(Lo) mice was protective, it is logical to suggest that the concomitant expression of Hi-FGF2 in excess of Lo-FGF2 in FGF2(WT) mice antagonizes or prevents protection by Lo-FGF2, by an as yet undetermined mechanism. Acute as well as sustained cardioprotection by extracellular-acting Lo-FGF2 is known to require FGF receptor 1 (FGFR1) (35) as well as the downstream activation of PKC-epsilon and protein kinase CK2 (36,37). Extracellular-acting Hi-FGF2 is believed to activate plasma membrane FGFR1 and downstream pathways in a manner broadly similar to Lo-FGF2(8,38). It is possible, however, that Hi-FGF2/FGFR1-triggered signaling pathways may not be identical to those triggered by Lo-FGF2/FGFR1, especially in the long-term. Binding of FGF2 isoforms to FGFR1 could elicit different receptor conformations culminating in some differences in downstream signaling. It is of relevance that Hi-FGF2 but not Lo-FGF2 prevents cell migration, a property requiring its unique amino-terminal extension which differentiates it from Lo-FGF2; in addition, Levin and colleagues have shown that the amino-terminal domain of Hi-FGF2 depends on an interaction with the co-receptor neuropilin 1 in order to inhibit cell migration (45). Thus, we suggest that different outcomes by paracrine/autocrine acting FGF2 isoforms may be caused by interaction with isoform-selective co-receptors, in addition to the isoform-non-selective FGFR1. Hi-FGF2 which exists in excess of Lo-FGF2 in vivo would be expected to occupy most of the plasma membrane FGF2 ligand binding sites, thereby favoring Hi-FGF2-driven signal transduction pathways, while reducing the contribution of Lo-FGF2/FGFR1-triggered signaling. Furthermore, FGF2 isoforms can become internalized and can also signal in an intracrine manner. We have shown that intracrine Hi-FGF2 can exert distinct, often deleterious effects, regardless of cell surface receptor occupancy; ectopic expression of Hi-FGF2 but not Lo-FGF2 caused myocyte binucleation (41), as well as mitochondrial damage, chromatin compaction, and myocyte cell death in vitro, and these effects were reversed by the pro-survival member of the Bcl-family, Bcl-2 (40). It is of interest that the ERK pathway, which is equally activated downstream of FGFR1 by Hi- and Lo-FGF2 (33), and which is considered cytoprotective and cardioprotective, can also mediate pro-apoptotic effects of intracellular Hi-FGF2 (40).

Taken together, our findings from the FGF2 mouse models indicate that exclusive expression of either isoform promotes early protection, but only Lo-FGF2 provides more sustained protection against Dox. At the same time, simultaneous expression of the Hi- and Lo-FGF2 isoforms in the wild type mice is not protective from Dox in comparison to the expression of either isoform alone. To explain these findings, we posit a theoretical model which states that although Hi- or Lo-FGF2 can both bind and activate the main FGF2 receptor in the heart and cardiomyocytes (19), FGFR1, differences may exist in some of the signaling pathways activated by the isoforms downstream or even independently of FGFR1; differences may also exist in how various cell types respond to single or combined FGF2 isoforms. Hi-, but not Lo-FGF2, is reported to engage the estrogen receptor in order to inhibit endothelial cell migration (24). It is likely that the net effect of signaling by physiological concentrations of Hi- and Lo-FGF2 combined ends up being less protective than each type of isoform acting alone, in vivo. In this model, increasing the relative levels of Lo-FGF2 (by exogenous supplementation; by overexpression) would shift the balance towards Lo-FGF2-dominated signaling and cardioprotection, as is in fact the case (7, 8, 21, 25). Another possibility we cannot exclude at this point is that Hi-FGF2 in FGF2(Hi) mice has a different biological activity than Hi-FGF2 in the FGF2(WT) mice, due perhaps to different post-translational modifications such as phosphorylation or methylation all of which are modifiers of the biological activity of FGF2 (22, 23).

The Role of Bnip3

Dox is a known mitochondrial toxin, impairing the integrity of the electron transport chain, altering its transcriptome, and forming mitochondrial permeability transition pores, leading to apoptosis and necrosis (26, 27). There is evidence that Bnip3, which is localized to cardiac mitochondria via its transmembrane domain, is central in Dox-induced mitochondrial perturbation and myocardial damage (28). Several in vivo studies have reported that Dox upregulated Bnip3 in the heart of male mice. In agreement, in our male groups, Dox did not upregulate cardiac Bnip3 protein in the ‘protected’ FGF2(Lo)mice, but did so in all the other groups; these findings suggested that cardioprotection in FGF2(Lo) mice was mediated, at least in part, by the lack of Bnip3 upregulation; conversely, Dox-induced deleterious effects in the other male groups could be attributed in part to Dox-induced Bnip3 upregulation. In in vitro studies, added Lo-FGF2 prevented the Dox-induced Bnip3 upregulation, in agreement with the in vivo experiments (8).

The relationship between FGF2 expression, Dox-induced Bnip3 upregulation, and cardiac damage appeared to be more complex in female groups. As was the case with male mice, Dox did not elicit Bnip3 upregulation in the ‘protected’ female FGF2(Lo) mice. However, unlike the situation with male groups, Dox did not upregulate Bnip3 in FGF2(WT) or FGF2(Hi) groups even though Dox caused decreased FS and EF. It would appear that, in females, expression of FGF2 regardless of isoform composition, acted as a deterrent against Dox-induced Bnip3 protein increases. Dox increased cardiac Bnip3 protein accumulation only in the FGF2(−) group, associated in this case with decreased FS and EF. It should be noted that relative Bnip3 protein levels are only indicative of possible involvement in the observed endpoint, and they may not be synonymous with activity. Bnip3 activity can be regulated by other factors, for example by post-translational modifications and/or interactions with other proteins.

FGF2 Expressed by Non-Myocytes Modulate Cardiomyocyte Resistance to Dox

Cardiac fibroblasts/myofibroblasts are a major cell population in the heart, and, through their paracrine action, integrate various pathological stimuli to cause adaptive and also maladaptive heart remodeling after various types of injury. Cardiac fibroblasts represent a major source of FGF2 produced in the heart (9, 10); previous studies have shown that lack of FGF2 expression by fibroblasts are likely responsible for the excessive dilatation in FGF2 knockout hearts after infarction or in response to angiotensin II-induced hypertension (17). We have previously shown that both Lo-FGF2 and Hi-FGF2 are secreted from normal rat or human fibroblasts and are present in conditioned media as well as biological fluids including pericardial fluid (9).

Previous studies have shown that lack of FGF2 expression in fibroblasts is responsible for the excessive dilatation in the hearts of mice depleted of all FGF2 after infarction or in response to angiotensin II (17). It was therefore reasonable to hypothesize that FGF2 expression by fibroblasts, in co-culture or fibroblast-conditioned medium experiments, could modulate the vulnerability of cardiomyocytes to a toxic agent, a hypothesis proven to be correct. We have previously shown that both Lo-FGF2 and Hi-FGF2 are secreted from normal rat or human fibroblasts and are present in conditioned media, in association with the extracellular matrix, as well as pericardial fluid (9). MEFs from FGF2(WT) and FGF2(Lo) mouse strains co-cultured with r-cardiomyocytes elicited distinct effects on cardiomyocyte vulnerability to Dox and, to some degree, recapitulated the in vivo phenotype as incubation with FGF2(Lo)-MEFs was protective. More specifically, this protection was measured by reduced cTnT release and increased mitochondrial calcein fluorescence in the attached cells, indicative of mitochondrial functionality and cell viability. At the same time, incubation with FGF2(WT) MEFs allowed Dox-induced toxicity to manifest, in agreement with our in vivo findings.

The use of co-culture systems, or mammalian fibroblast conditioned media, are useful approaches to study the effects of secreted fibroblast proteins at near-physiological concentrations, in the presence not only of the protein of interest (in this case FGF2) but all other factors secreted at the same time.

Different approaches such as the addition of supra-physiologic levels of recombinant proteins, or ectopic overexpression, do not necessarily inform on the endogenous role of the proteins under investigation. MEFs from the different mouse strains, co-cultured with rat neonatal cardiomyocytes for two days, elicited distinct effects on cardiomyocyte vulnerability to Dox, and, to some degree, recapitulated the in vivo phenotype, since incubation with FGF2(Lo)-MEFs was shown to be protective, as measured by reduced Troponin T (Tn-T) release to the supernatant, and increased calcein fluorescence in the attached cells, indicative of mitochondrial functionality. At the same time, incubation with FGF2(WT)-FGF2(−) and FGF2(Hi)-MEFs allowed Dox-induced toxicity to manifest, in agreement with our in vivo findings at the 8-10 days post-Dox time point.

In the Wild Type Background, Blocking Hi-FGF2 with a Neutralizing Antibody Mitigates Dox Toxicity in Cardiac Myocytes Exposed to Secreted Human FGF2

We have provided strong evidence that concomitant expression of FGF2 isoforms in vivo and in vitro renders cardiomyocytes more vulnerable to Dox, compared to sole expression of Lo-FGF2. Hence, we postulated that targeting Hi-FGF2 selectively by a neutralizing antibody could protect cardiomyocytes against Dox insult. A neutralizing antibody specific for human Hi-FGF2 (Hi-Ab) was able to attenuate cardiomyocyte Dox-induced damage r-cardiomycetes in three different scenarios: (1) cardiomyocytes cultured with human fibroblasts plated on permeable Transwell membranes, (2) co-cultures of cardiomyocytes with human fibroblasts, or (3) application of conditioned media from human fibroblasts on cardiomyocyte monolayers. Importantly, in the #3 scenario, use of neutralizing antibodies for total FGF2 did not exert protection but rather increased vulnerability to Dox, presumably by blocking the action of Lo-FGF2 in addition to Hi-FGF2.

It was noted that when using conditioned medium from Fibs^{iPSC}, relative damage by Dox, as well as protection by Hi-Ab, appeared less pronounced than when Fibs^{iPSC} are co-plated with myocytes. It is reasoned that in direct co-cultures the relative levels of paracrine human FGF2 (dominated by Hi-FGF2) are higher than those in conditioned medium alone, because they include not only FGF2 present in the soluble phase (conditioned medium) but also cell-released human FGF2 that is sequestered by the extracellular matrix. Our previous studies have demonstrated that human fibroblast-secreted FGF2 is detected in both conditioned medium and also amongst proteins bound to the cell surface/extracellular matrix (9). Higher relative levels of paracrine Hi-FGF2 (in solution and in association with the matrix) would be expected to be more deleterious to Dox-treated myocytes, and at the same time allow a larger range against which to measure protection by Hi-Ab. Variations in culture medium volume may also contribute to dilution of signal for Dox-induced cTnT release in the Transwell set-up. Regardless however of the magnitude of damage and protection, all in vitro experiments show qualitatively similar results namely that paracrine-acting FGF2 produced by fibroblasts influences r-cardiomyocyte vulnerability to Dox and that, in a wild-type environment, neutralization of paracrine Hi-FGF2 raises cardiomyocyte resistance to Dox, likely by allowing unopposed action of Lo-FGF2.

We have documented that exclusive expression of Lo-FGF2 in vivo is cardioprotective, preventing Dox-induced contractile dysfunction and cardiac dilatation and Dox-induced cardiac deterioration. Our in vitro studies support the in vivo observations and produced the novel and important finding that cardiomyocyte protection from Dox can be promoted via selectively neutralizing Hi-FGF2 in a wild-type paracrine environment. Antibody-based approaches are widely used in humans to neutralize the activity of ligands or receptors via systemic administration. Thus, an antibody-based approach to target paracrine Hi-FGF2 in the extracellular matrix in vivo would be expected to confer cardioprotection against genotoxic damage as well as other injurious stimuli.

Cardiomyocyte protection via selectively targeting Hi-FGF2 in a wild type environment is a novel finding, and of clinical relevance as it represents a prophylactic treatment for patients receiving anthracyclines.

As will be appreciated by one of skill in the art, the FGF2 protein has traditionally been considered to be cardioprotective. As such, one of skill in the art would assume that elimination or neutralization or otherwise reducing the amount of FGF2 would hurt the heart, making it more vulnerable to toxic stimuli. As discussed herein, that is exactly what happens when both forms of FGF2 are eliminated. However, the fact that an anti-HI-FGF2 antibody increases protection is counter-intuitive and surprising.

The invention will now be further elucidated and explained by way of Examples. However, the invention is not necessarily limited to the examples.

### EXAMPLES

With every heart-beat, blood is pumped to the body to meet the ongoing oxygen and nutrient requirements of the body organs. In healthy individuals at rest, the heart pumps out blood with a force at baseline levels; when the individual's activity increases, the heart increases its pumping power to meet the increased needs. The term ‘Ejection Fraction, EF, is a measure of the pumping ability of the heart, and is measured by ultrasound (Echocardiography). EF represents the percentage of the blood pumped out from the heart at the time of measurement. In case of heart stress and damage the heart's ability to pump blood is compromised/decreased and this is measured as a decrease in EF values by echocardiography. In short, a drop in EF suggests that the heart contractile function is damaged, which may be the result of heart muscle injury.

If the heart is diseased (possibly by formation of scar tissue, or fibrosis, and/or a weakening of muscle strength), it presents reduced ability to pump blood out of the heart effectively. As a consequence, there is residual blood inside the heart which increases the size and pressure in the heart (heart dilatation). The internal size of the heart can be measured by echocardiography, and one of the parameters measured is termed LVEDD (left ventricular end diastolic diameter). When the heart is dilated, LVEDD is higher than normal, and increased LVEDD beyond certain limits signals the onset of heart failure. In our IN VIVO study we have observed that the chemotherapy drug, Dox, decreased EF and increased LVEDD in male wild type mice, meaning that Dox caused cardiac damage leading to heart failure. However, in the genetically engineered male mice where Hi-FGF-2 was deleted genetically, no decrease in EF or increase in LVEDD was observed, meaning that the hearts of those animals were protected against Dox.

As discussed herein, our work with heart cells in culture allowed to show that our reagent (Hi-Ab) has the ability to ‘neutralize’ Hi-FGF2, and protect heart muscle cells from Doxorubicin.

### Example I—Baseline Characteristics of the FGF2 Mouse Models

FGF2(−), FGF2(Hi), and FGF2(Lo) genetically engineered mice have been characterized previously (11-13). With the exception of FGF2(−) lacking all FGF2, the remaining groups expressed comparable levels of total FGF2, regardless of isoform composition. All groups had normal pregnancies and did not display any gross morphological abnormalities. Differences were observed in baseline body weights. In males, FGF2(Lo) mice had smaller body weight, by 8%, compared to the FGF2(WT) group, while the FGF2(−) and FGF2(Hi), groups were heavier, by 12% and 15%, respectively, compared to FGF2(WT). A broadly similar pattern was observed amongst the female groups. Thus, the absence of Lo-FGF2, in the FGF2(−), and FGF2(Hi) groups was associated with overall heavier body weights.

Baseline echocardiography measurements indicated that systolic function and LVEDD were within the normal range in all groups both males and females.

### Example II—the Effect of Dox on Mortality and Body Weight

Dox-induced mortality was highest (70%) in male FGF2(−) mice, followed by female FGF2(−) mice at 30%, by day 10 post-Dox. No deaths occurred in any of the remaining female groups, or in any of the sham groups. The post-Dox mortality of male FGF2(Hi), and male FGF2(WT) was at 20% and 10%, respectively. Dox induced some weight loss in all groups.

### Example III—Endogenous FGF2 Expression and Vulnerability to Dox

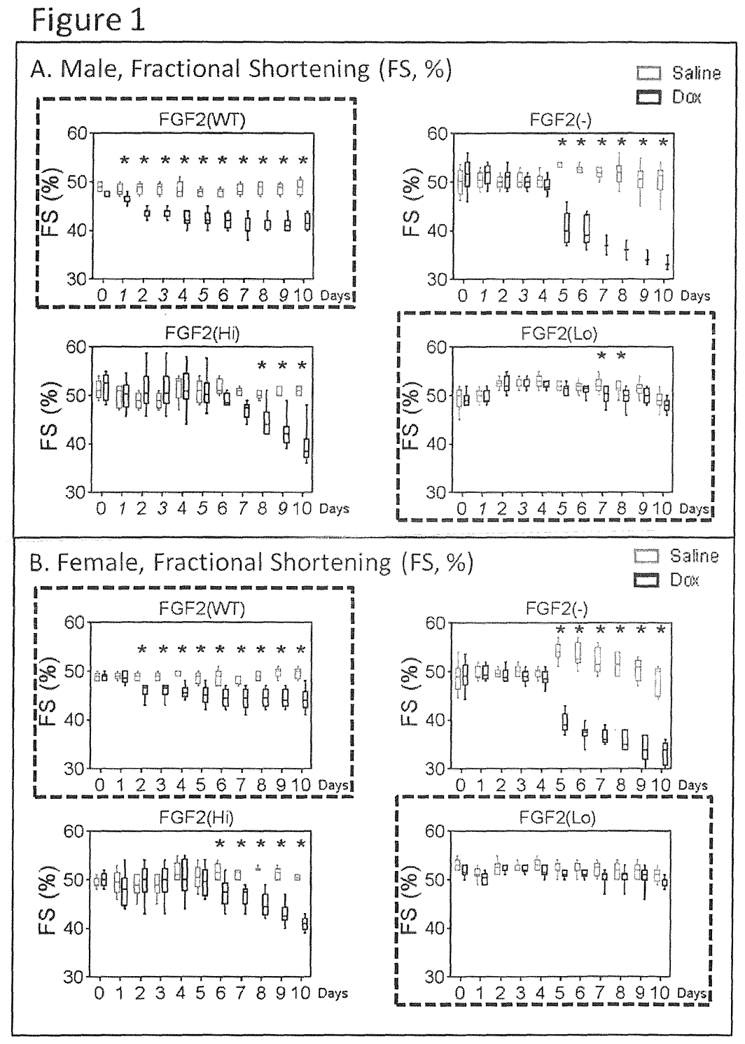

FIG. 1 shows the effect of FGF2 expression on cardiac fractional shortening (FS) post-Dox versus post-saline injection. FS was measured daily. Each Dox-treated group is compared to its corresponding sham (saline-injected) control at the same time point. In males (FIG. 1A), the FGF2(WT) group showed a progressive decrease in FS starting from day 2 (by 11%) and progressing to 15% by day 10. FS in the FGF(−) group was not affected up to 4 days post-Dox, but declined abruptly after day 5, reaching a 34% decrease by day 10, substantially more severe (>2-fold) than that in the FGF2(WT) group. The abrupt decline in FS at 5-6 days post Dox coincided with 40-70% mortality in this group during these time points.

Thus, this figure shows a measure (FS) of cardiac functionality in terms of pumping ability. Decreases from the normal values reflect functional decline. For example, compare what happens in FGF2(WT) versus FGF2(Lo), where only the second group shows preservation of functionality after 10 days. The FGF2(Hi) group was protected from decreased FS up till day 7 post-Dox, but reached a 22% decrease by day 10. The FS decline in the FGF2(Hi) group appeared intermediate between the FGF2(−) and FGF2(WT) groups, suggesting that the presence of some Lo-FGF2 in the FGF2(WT) might have exerted some protection.

The FGF2(Lo) group showed no decrease in FS by day 10 post Dox. The female groups displayed a similar pattern of FS responses as their male counterparts (FIG. 1B). In the case of the FGF2(−) females, mortality at 5-6 days post Dox was, at 10%, substantially less severe than that of their male counterparts at the same time points, despite similar magnitude in FS decline.

As will be appreciated by one of skill in the art, FS reflects the hearts contractility and a decline in FS means cardiac damage. Here, we have observed that FS declined in FGF2(WT) mice since day 1, while it was unchanged in FGF2(Lo) animals, meaning that elimination of Hi-FGF-2 protected hearts against Dox damage.

In summary, we observed that while doxorubicin caused a decline in FS in normal animals (FGF2(WT) that produce both Hi- and Lo-FGF2, it did not do so in animals where Hi-FGF2 had been genetically eliminated, leaving only the Lo-FGF2 isoform, FGF2(Lo) mice. Our data indicate that in normal animals Hi-FGF2 has a detrimental effect on FS, while Lo-FGF2 is protective.

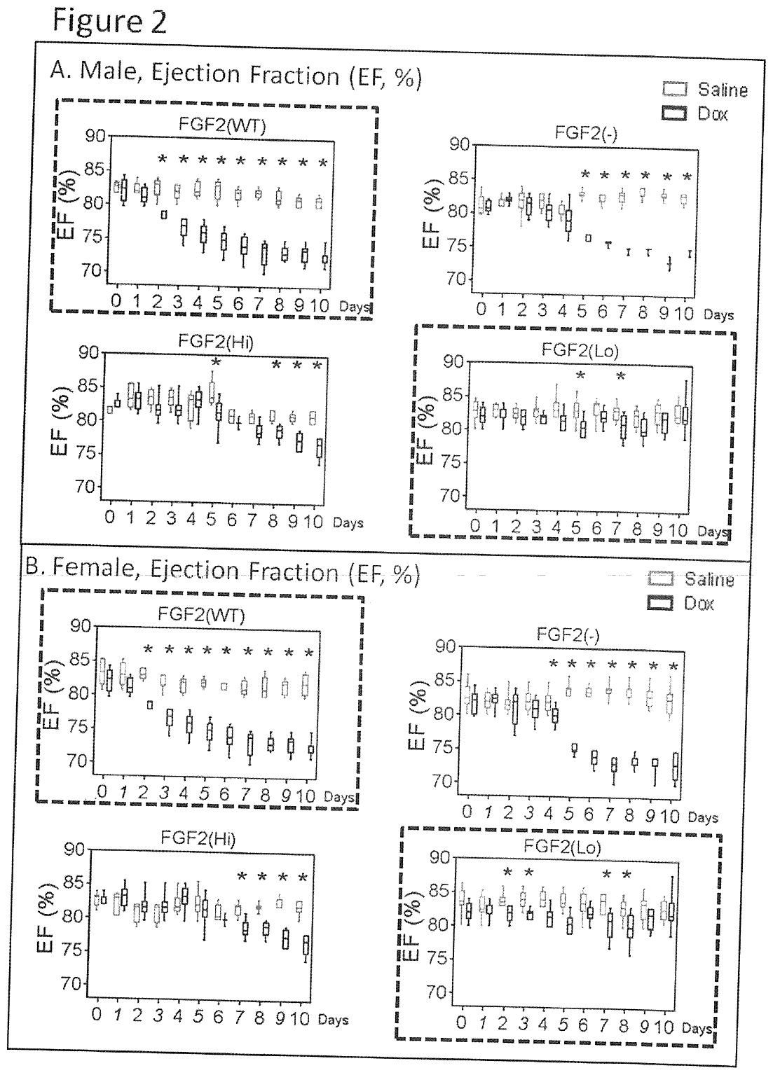

FIG. 2 shows the effect of FGF2 expression on cardiac ejection fraction (EF) post-Dox (or post-saline) in male and female groups. In males, the wild type FGF2(WT) group (FIG. 1, A) displayed an early, starting at day 2 post-Dox, decrease in EF (by 5%), which reached a 10% decrease by day 10. The FGF2(−) group did not show any decrease in EF for up to 4 days post-Dox. A decrease in EF was detected at days 5-10, reaching a maximum of 10%. FGF2(Hi) mice appeared to be protected from a decline in EF up to day 7; a 5% decrease was however evident by day 10. By day 10 no differences in EF were observed between sham and Dox-treated groups. The female groups displayed similar patterns of EF changes post-Dox as their male counterparts (FIG. 1B).

As discussed herein, our data indicate that in normal animals Hi-FGF2 has a detrimental effect on EF, while Lo-FGF2 is protective.

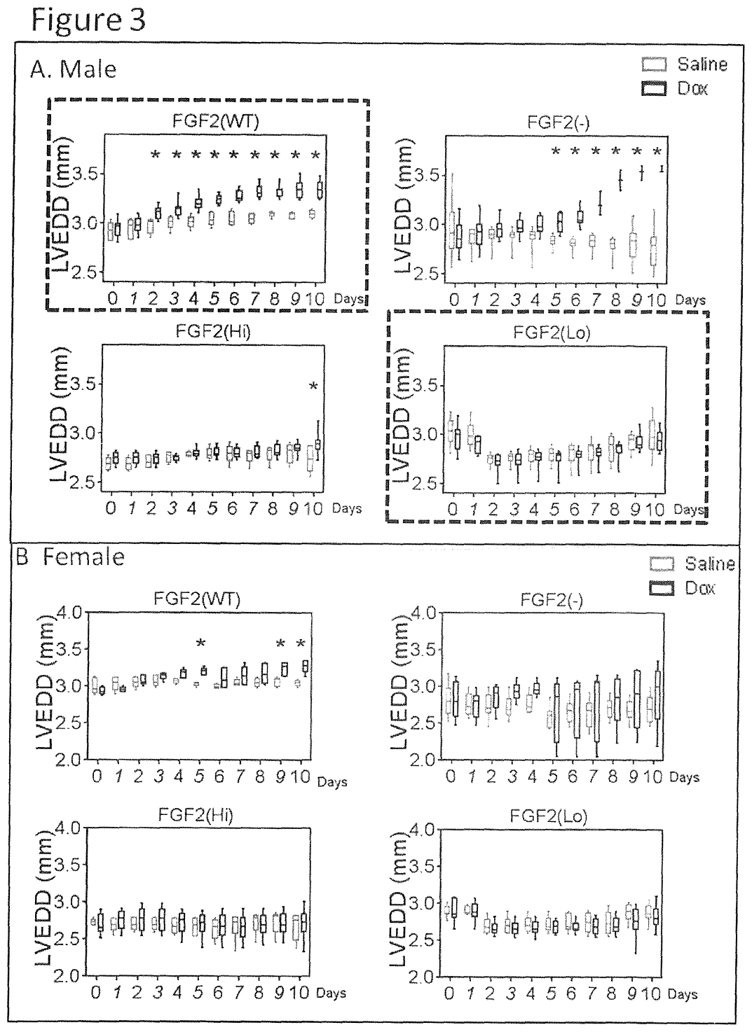

FIG. 3 shows the effect of FGF2 expression on left ventricular end diastolic diameter, LVEDD, post-Dox. LVEDD is an indirect measure of the left intraventricular pressure and relates to myocardial deformation and stiffness; increases in LVEDD indicate pathological changes such as cardiac dilatation. In males, FIG. 3A, FGF2(WT) mice showed a small, progressive increase in LVEDD post-Dox, starting at day 2, by 4%, and reaching 8% increase at day 10. LVEDD in FGF2(−) mice did not change for up to 4 days post-Dox; a significant 7% increase was observed at day 5, reaching 29% increase by day 10. The FGF2(Hi) mice showed a 6% increase in LVEDD by day 10. No changes were seen in the FGF2(Lo) group at any time point.

The pattern of LVEDD responses in the female groups did not parallel that of the male groups, and would be consistent with an overall protection from Dox-induced LVEDD increases. None of the female groups (FIG. 3B) showed any changes in LVEDD for up to 4 days post-Dox.

Subsequently, only the FGF2(WT) group presented a modest 7% increase by day 10. Female FGF2 (−) mice showed no increases in LVEDD post-Dox, in sharp contrast to the 29% increase seen in males. FGF2(Hi) and FGF2(Lo) female groups were protected from increased LVEDD post-Dox.

In our study we have observed that the chemotherapy drug, Dox, increased LVEDD in male wild type mice, meaning that Dox caused cardiac damage leading to heart failure. However, in the genetically engineered male mice where Hi-FGF-2 was deleted, leaving only Lo-FGF2, no increase in LVEDD was observed, meaning that the hearts of those animals were protected against Dox.

### Example IV—Bnip3 Protein Expression

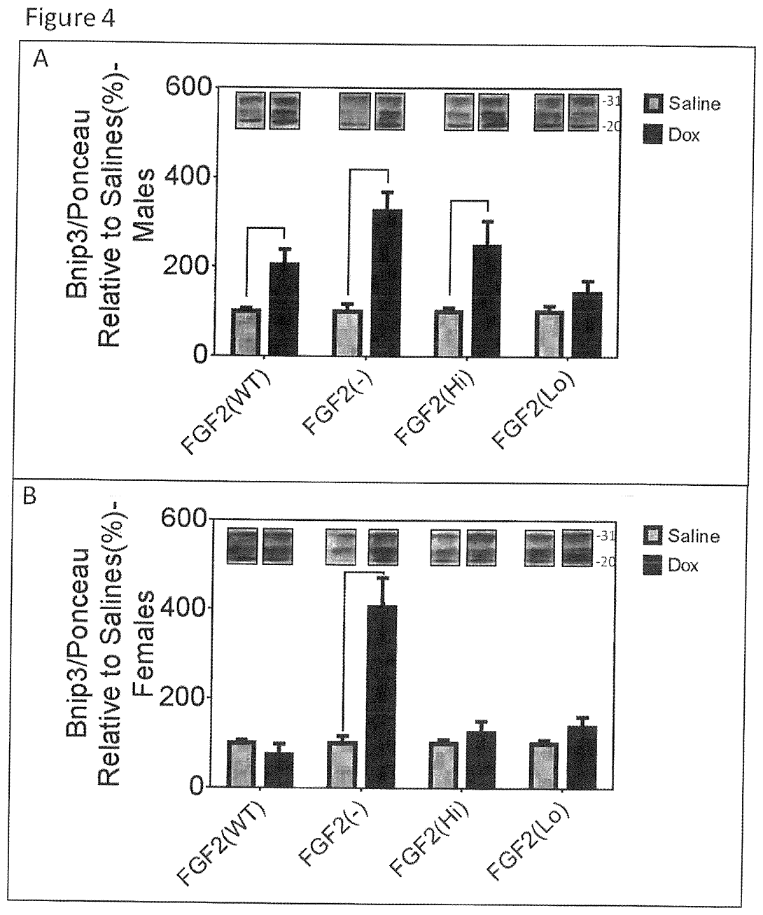

The Bnip3 protein is implicated in Dox-induced mitochondrial damage leading to cell death and cardiac deterioration (14). In males, Dox significantly upregulated relative Bnip3 levels in hearts of FGF2(WT), FGF2(−), and FGF2(Hi), but not FGF2(Lo), mice compared to sham groups, as shown in FIG. 4A. In females, Bnip3 was only increased in the FGF2(−) mice post-Dox (FIG. 4B). Baseline (untreated) relative Bnip3 protein levels were similar between all groups.

### Example V the Effects of Mouse Embryonic Fibroblast (MEFs) on Cardiomyocyte Resistance to Dox

To examine the role of non-myocyte FGF2 expression on cardiomyocyte resistance to injury, we co-cultured rat cardiomyocytes with MEFs derived from FGF2(+), FGF2(−), FGF2(Hi) and FGF2(Lo) mice, for two days, and then exposed them to Dox. Rat ventricular cardiomyocytes do express FGF2 (Hi- and Lo-), at lower levels that non-myocytes, as we have shown (10). As a consequence, it is expected that the total secreted FGF2 would largely reflect the MEF-originating contribution.

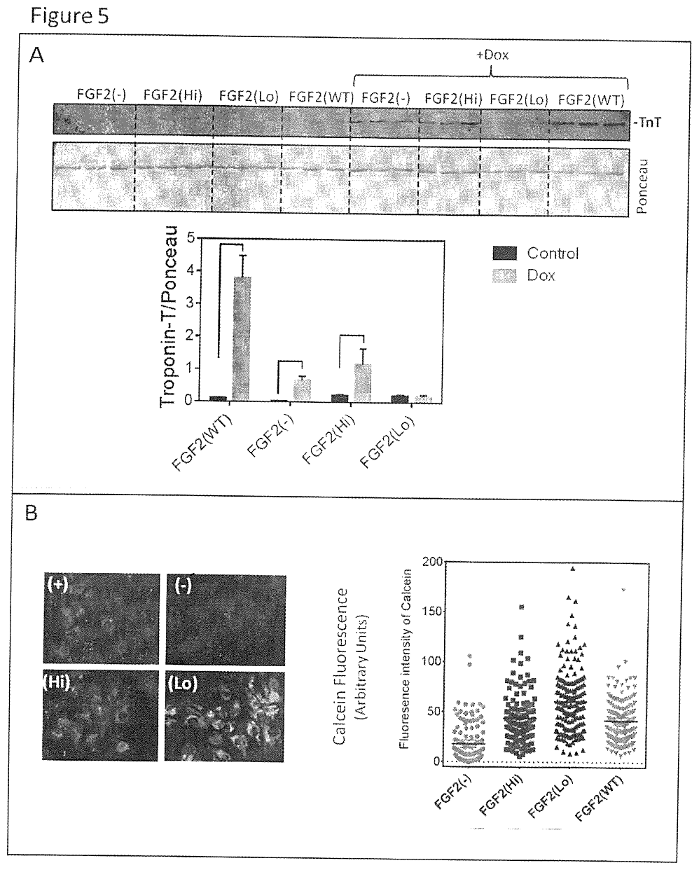

Cardiomyocyte damage was detected by probing for cardiac Troponin T (Tn-T), expressed only by cardiomyocytes, in the supernatant. As seen in FIG. 5A, Dox-induced Tn-T release was observed in co-cultures with FGF2(WT), FGF2(−), and FGF2(Hi) MEFs, but not with FGF2(Lo) MEFs. Dox triggers mitochondrial permeability transition leading to cell death. We used a Calcein AM-cobalt chloride assay kit on live cells (in co-cultures) to visualize healthy mitochondria and viable cells post-Dox. Because cardiomyocytes are packed with mitochondria, they are expected to be the major contributors to the calcein fluorescence signal. As shown in FIG. 5B, relative intensity of calcein fluorescence was significantly higher in co-cultures with FGF2(Lo)-MEFs, compared to co-cultures with FGF2(WT)-, FGF2(Hi)-, or FGF2(−)-MEFs. Thus, the MEF co-culture findings are in broad agreement with the in vivo mouse data presented in FIGS. 1-3, and support the notion that the composition of FGF2 expressed by non-myocytes plays an important role in modulating cardiomyocyte vulnerability to Dox injury. Our findings also suggest that in the physiological milieu, the simultaneous expression/secretion of Hi- and Lo-FGF2, at the 7:3 ratio determined by our previous studies (9, 10) is not, compared to secretion of Lo-FGF2 alone, protective from Dox injury. Because co-culture with FGF2(Lo) MEFs was protective, we hypothesized that in a wild-type background, selective neutralization of Hi-FGF2 produced by non-myocytes would be beneficial to cardiomyocytes, by allowing ‘unopposed’ protective action of Lo-FGF2.

It is known that in vivo cells from connective tissue (fibroblasts) produce most of the FGF2 protein which can then act on the muscle cells. Here we cultured fibroblasts from the different FGF2 mice (having different FGF2 profiles) together with normal cardiac muscle cells, and then added Doxorubicin. To measure how much damage the muscle cells suffer, we measured how much heart-muscle-specific-protein is released from muscle cells into the environment (this simulates Troponin measurements in cardiac patient blood, in the clinic). The main message in FIG. 5 is that muscle cells cultured together with fibroblasts that can only produce Lo-FGF2 (FGF2(lo) MEFS) suffer less damage from Doxorubicin, compared to those cultured with fibroblasts from all other FGF groups.

### Example VI—the Effects of (Human) Hi-FGF2-Specific Antibodies on Dox-Induced Cardiomyocyte Damage In Vitro

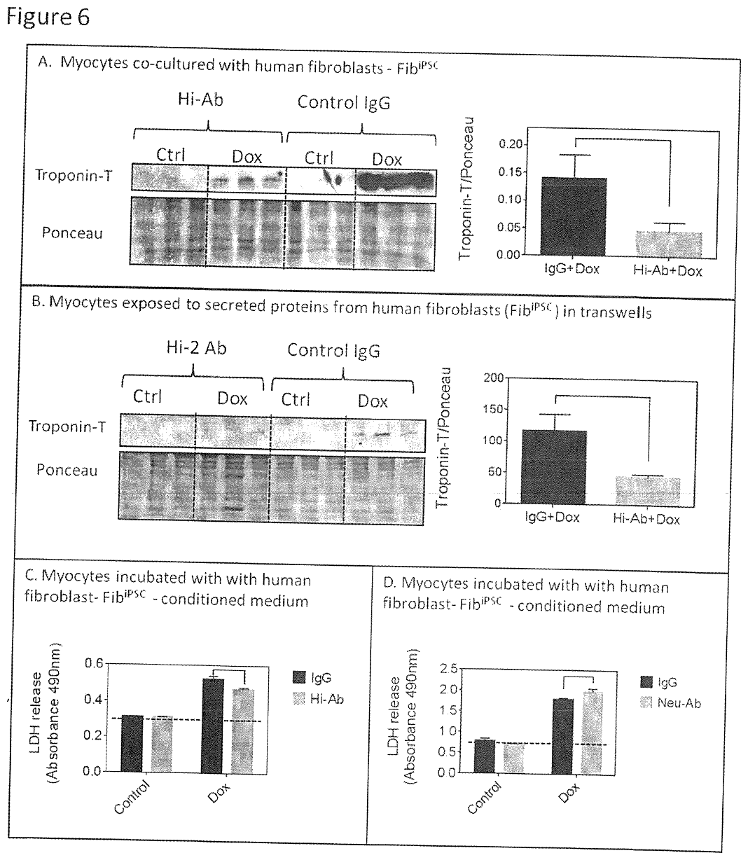

Hi-FGF2 isoforms possess an extra N-terminal extension which is absent from Lo-FGF2(15). We have obtained and characterized affinity-purified antibodies against a sequence within the N-terminal extension of human Hi-FGF2, Hi-Ab, as we described in (9); and as described by others in (16). Human fibroblasts, derived from induced pluripotent stem cells (Fib^{iPSC}) were used as a source of human non-myocyte FGF2. Both types of isoforms are expressed by Fib^{iPSC}; Hi-Ab interacted with, and immunoprecipitated, native human Hi-but not Lo-FGF2 from Fib^{iPSC}. Hi-Ab do not recognize rat Hi-FGF2 (9), so they cannot neutralize rodent Hi-FGF2. Cardiomyocytes, co-cultured with Fibs^{iPSC}, were exposed to Dox, in the presence of Hi-Ab or, in control cultures, non-immune rabbit immunoglobulin. As shown in FIG. 6A, Dox promoted cardiomyocyte damage (Tn-T) release in control co-cultures. Damage was significantly attenuated in the presence of Hi-Ab, suggesting that neutralization of secreted human Hi-FGF2 was protective. In a parallel experiment (FIG. 6B), cardiomyocytes were separated from Fib^{iPSC} by plating the latter in Transwell permeable inserts, in the presence of Hi-Ab or control IgG. The presence of Hi-Ab resulted in reduced Tn-T release in response to Dox.

In a similar manner, when cardiomyocytes were exposed to conditioned medium from Fibs^{iPSC}, Dox elicited less damage (LDH release) in the presence of Hi-Ab, FIG. 6C. In a separate experiment, cardiomyocyte incubation with human Fib^{iPSC} conditioned medium in the presence of antibodies capable of interacting with, and neutralizing, total FGF2 (both Hi- and Lo-FGF2), had the opposite effect, increasing Dox-induced damage, FIG. 6D. Taken together, the in vitro studies showed that: FGF2 expressed by fibroblasts modulates cardiomyocyte vulnerability to Dox; direct contact/interaction between myocyte and non-myocytes is not necessary for the observed effect which is mediated by secreted FGF2; neutralization of secreted Hi-FGF2 produced by non-myocytes in a wild type environment raises cardiomyocyte resistance to Dox, via Lo-FGF2.

As such, we have now tested if, by blocking Hi-FGF2 present in the environment of cardiac muscle cells, we could prevent Doxorubicin damage. We used human-type fibroblasts as Hi-FGF2 producers, because our reagent (Hi-AB) is specific for human Hi-FGF2. Three different experimental setups (A,B,C) were used to expose normal cardiac muscle cells to human FGF2 (consisting of Hi- and Lo-FGF2 types) as produced by human fibroblasts. When they were then exposed to doxorubicin, damage was less severe if cardiomyocytes were pre-treated with Hi-Ab. That's because with neutralization of Hi-FGF2, the remaining Lo-FGF2 is able to exert its protective effect.

In an additional testing (D), we show that if we neutralized all FGF2 present in the cultures, we ended up increasing damage of muscle cells. That's because both Hi- and Lo-FGF2 action was prevented and therefore Lo-FGF2 could not exert protective effects.

Endogenous FGF2 Expression and Vulnerability to Dox.

Baseline characteristics of the FGF2(Lo) mouse model have been reported previously (13). Echocardiographic measurements indicated that that systolic function and left ventricular cavity dimensions were within the normal range for all groups of male and female mice.

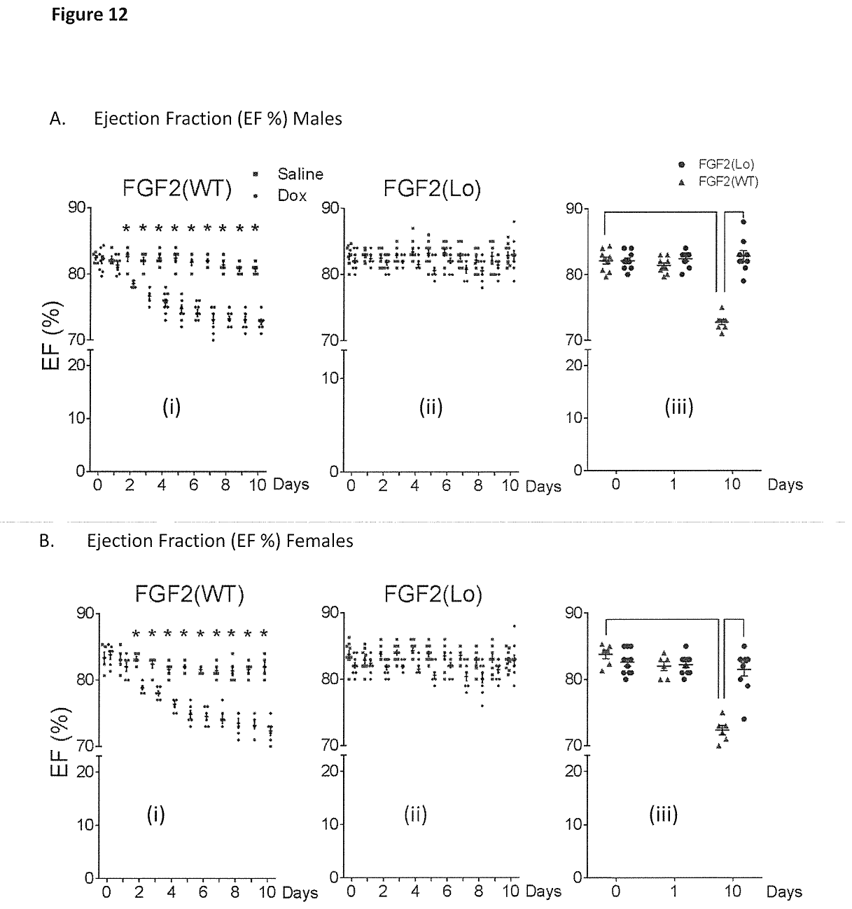

Dox had minimal effects on mortality in FGF2(Lo) and FGF2(WT) mouse models. In males, Dox administration was associated with one death in ten animals, in either the FGF2(WT) or the FGF2(Lo) groups. No death occurred in any of the female groups. The effect of Dox- or saline-injection on ejection fraction (EF) for up to 10 days post-Dox treatment in FGF2(WT) and FGF2(Lo) mice is shown for males (FIG. 12A) and females (FIG. 12B). In FIG. 12A or 12B, panels (i) and (ii), comparisons are made between the Dox- and corresponding saline-treated (sham) animals at each time point. In males, the wild type FGF2(WT) mouse group, FIG. 12A, panel (i), displayed a gradual decrease in EF, compared to sham, reaching 10% decrease by day 10. The Dox-treated FGF2(Lo) group did not show a decline in EF, compared to sham, FIG. 12A, panel (ii). As shown in FIG. 12B (panels (i) and (ii)) the female groups had similar patterns of EF changes post-Dox as their male counterparts. EF was also compared between FGF2(WT) and FGF2(Lo) mice at baseline and 10 days post-Dox treatment, shown in FIG. 12A or 12B, panel (iii). While no significant differences were seen at baseline, at 10 days post Dox treatment the EF values of male as well as female FGF2(WT) groups were significantly lower (by 12% and 11%, respectively) compared to corresponding FGF2(Lo) mouse groups.

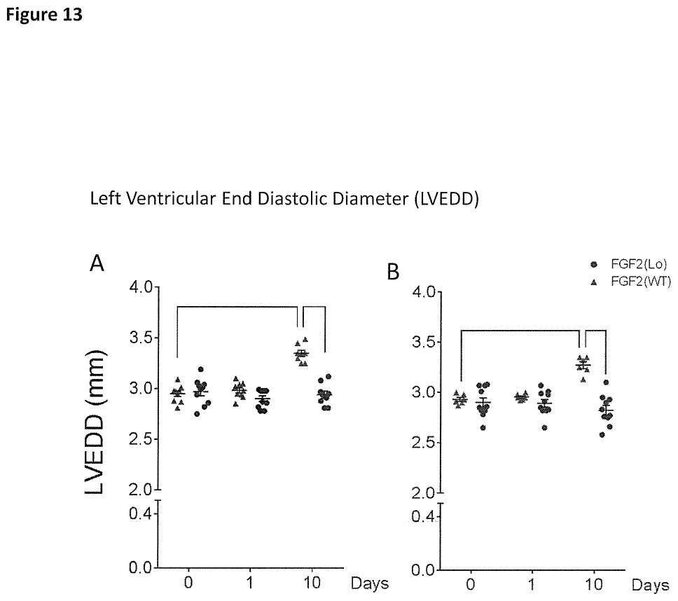

Measurements of left ventricular end diastolic dimension, LVEDD at baseline, one day- and 10 days post-Dox treatment, in FGF2(WT) versus FGF2(Lo) mice, in males and females, are shown in FIG. 13. There were no differences between FGF2(WT) versus FGF2(Lo) mouse groups at baseline or at one day post-Dox treatment in either males or females. At 10 days post-Dox, LVEDD in the FGF2(WT) mice was higher, by 14% in males or by 16% in females, compared to the corresponding FGF2(Lo) mouse groups (FIG. 13).

Overall, the FGF2(WT) mouse groups showed Dox-induced deterioration of cardiac parameters (based on EF and LVEDD measurements) which were not observed in the FGF2(Lo) mouse groups. Additional data, from echocardiography and heart weight/body weight (HW/BW) parameters, at day 10 post-Dox or post-saline administration, are summarized in Table 1. The FGF(WT) but not the FGF(Lo) groups displayed a significant increase in left ventricular end systolic diameter, LVESD, compared to the corresponding saline group, but no changes in posterior wall thickness, PWT, or heart rate, HR, post-Dox in either males or females. In addition, no significant differences in HW/BW were observed between groups. No sex-related differences were evident in any of these measurements. Pilot studies using frozen cardiac ventricular sections from the various groups, obtained at 10 days post-saline or post-Dox, did not show evidence for apoptosis, myocardial damage or fibrosis, assessed, respectively, by: TUNEL staining; eosin/hematoxylin staining; Picrosirius Red staining, and were not pursued further. Based on previous work (34) in a similar model, it is assumed that any Dox-induced cell death and tissue damage might have been detectable at earlier time points, such as days 3-7 post-Dox, but not necessarily later on.

Accumulation of the Pro-Cell Death Protein Bnip3.

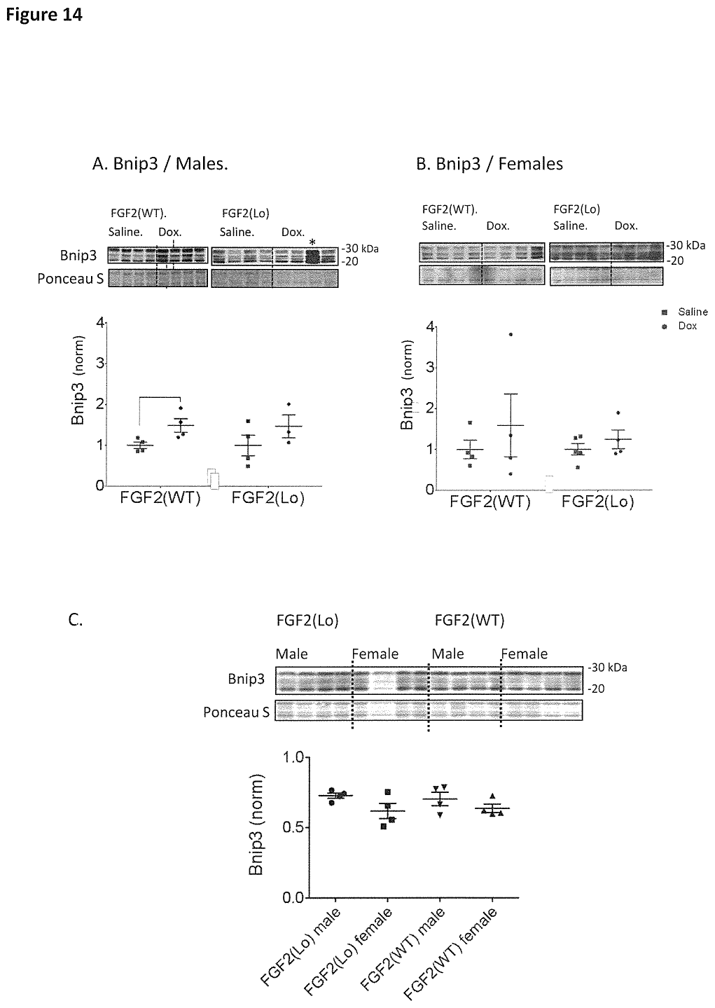

Increases in the pro-cell death protein Bnip3 (BCL2/adenovirus E1B 19 kDa protein-interacting protein) are implicated in Dox-induced mitochondrial damage and dysfunction, leading to cell death and cardiac deterioration (28). Therefore, Bnip3 protein expression was examined to determine whether it would parallel the functional differences observed between the (WT) versus (Lo) groups post Dox treatment (FIG. 14A,B). Baseline Bnip3 protein levels were similar between all groups (FIG. 14C). Please note that the anti-Bnip3 antibodies recognized several immunoreactive bands at 21 to 30 kDa, exactly as reported by others for MEFs, or rat cardiac ventricles (43,44). The anti-Bnip3 21-30 kDa immunoreactive bands were used for relative quantification of Bnip3 at day 10 post-treatment. The western blot data suggested that, in males, Dox upregulated cardiac Bnip3 protein in FGF2(WT) by 1.5-fold versus the saline-treated group (FIG. 14A). No significant Bnip3 increase was observed in the FGF2(Lo) mice compared to their corresponding sham groups. In females, Dox did not seem to increase cardiac Bnip3 significantly in either FGF2(WT) or FGF2(Lo) mice compared to their corresponding sham controls, FIG. 14B, suggesting the possibility of sex-related differences in Dox-induced Bnip3 response in wild type mice. Further studies are required to investigate this issue.

The Effects of FGF2 Expression by MEFs on r-Cardiomyocyte Resistance to Dox.

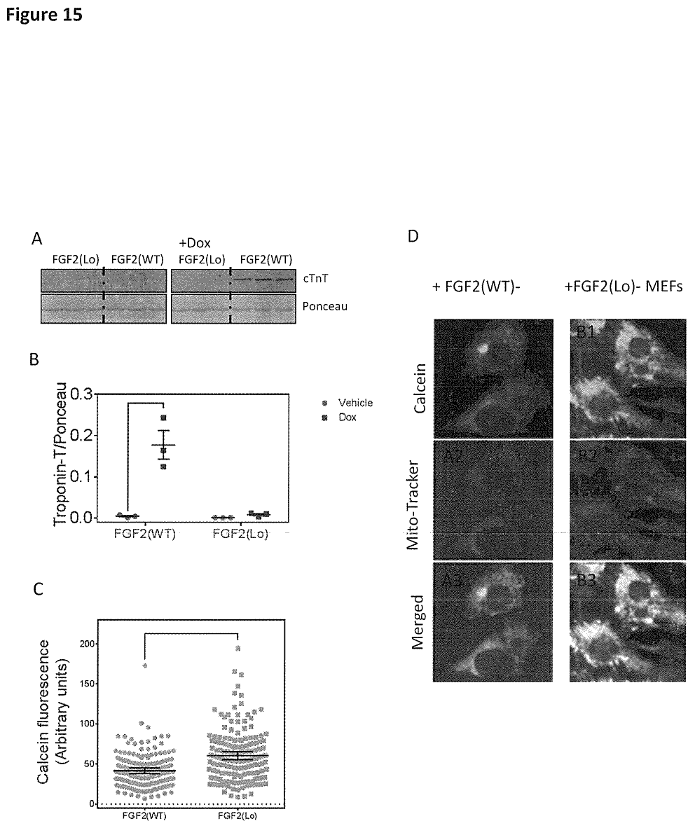

To examine the role of FGF2 produced by non-myocytes on cardiomyocyte resistance to injury, r-cardiomyocytes were co-cultured with MEFs derived from FGF2(WT) and FGF2(Lo) mice for two days and then they were treated with Dox. Note, r-cardiomyocytes express negligible levels of FGF2 protein relative to non-myocytes, as reported for both neonatal and adult cell lysates analyzed by protein (western) immunoblotting, as well as by immunostaining of adult hearts (10). Thus, it is expected that the total secreted/extracellular FGF2 in the co-cultures would largely reflect contribution from MEFs. R-cardiomyocyte-specific damage was detected by probing for cardiac cTnT in the culture medium. Dox-induced cTnT release was observed in co-cultures with FGF2(WT) but not with FGF2(Lo) MEFs (FIG. 15A). Dox triggers formation of mPTPs leading to cell death (39). A Calcein AM-cobalt chloride assay kit was used on live cells (in co-cultures) to visualize healthy mitochondria and viable cells post-Dox treatment. As mitochondria are more abundant in cardiomyocytes than in fibroblasts, myocyte mitochondria are the main contributors to the calcein fluorescence signal. The relative intensity of calcein fluorescence of r-cardiomyocytes exposed to Dox was significantly higher in co-cultures with FGF2(Lo)-MEFs, compared to co-cultures with FGF2(WT)-MEFs (FIG. 15B). Thus, the MEF co-culture findings are in broad agreement with the in vivo mouse data presented here (FIGS. 12 and 13). Furthermore, these observations are consistent with the notion that the isoform composition of FGF2 expressed by non-myocytes is playing a crucial role in determining cardiomyocyte vulnerability to Dox injury. These findings also suggest that the simultaneous presence of Hi- and Lo-FGF2 in the extracellular milieu, at the 7:3 ratio determined by our previous studies (9,10), is not, compared to secretion of Lo-FGF2 alone, protective from Dox injury. If so, selective neutralization of paracrine Hi-FGF2 is predicted to be beneficial to cardiomyocytes with a wild-type FGF-2 background, by allowing ‘unopposed’ protective action by Lo-FGF2.

The Effects of (Human) Hi-FGF2-Specific Antibodies on Dox-Induced r-Cardiomyocyte Injury In Vitro.

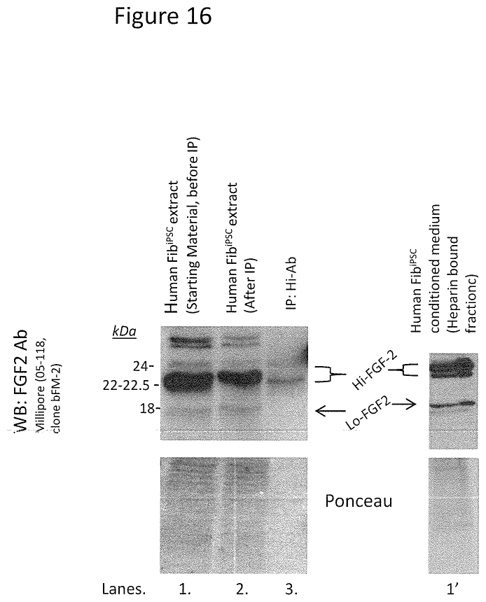

Hi-FGF2 isoforms possess an extra amino-terminal extension which is absent from Lo-FGF2(15). To selectively target Hi-FGF2, and discriminate between the isoforms, affinity-purified antibodies (Hi-Ab) were generated against a sequence in the amino-terminal extension of human Hi-FGF2 (9,16). Hi-Ab was shown in our previous studies to selectively neutralize the activity of extracellular-acting human Hi-FGF2 produced by adult human fibroblasts in vitro (9). The human fibroblasts used here as a source of paracrine-acting Hi-FGF2 were derived from induced pluripotent stem cells (h-Fib^{iPSC}). Both types of isoforms are expressed by, and are exported to the extracellular space of, h-Fib^{iPSC}, FIG. 16. As shown in FIG. 16 (lanes 1-3), Hi-Ab interacts with and immunoprecipitated native human Hi-but not human Lo-FGF2 from h-Fib^{iPSC}. In the example shown in FIG. 16, Hi-Ab immunoprecipitated about 40% of the soluble Hi-FGF2; similar findings have been reproduced twice using lysates from different batches of cells. It is surmised that, by interacting with extracellular human Hi-FGF, Hi-Ab decrease the relative levels of free human Hi-FGF2 available to interact with r-cardiomyocyte cell surface receptors in our co-cultures. In addition, because Hi-Ab does not recognize and cannot neutralize rat Hi-FGF2, as demonstrated in previous studies (9), any observed effect by Hi-Ab can be attributed to the neutralization of human Hi-FGF2 produced by h-Fib^{iPSC}.

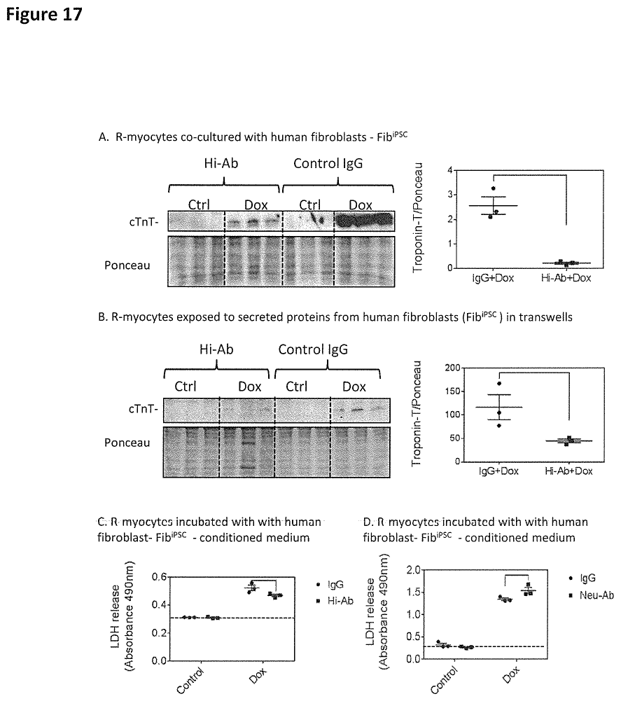

R-cardiomyocytes were co-cultured with h-Fibs^{iPSC} followed by exposure to Dox in the presence of Hi-Ab or, as a control, non-immune rabbit immunoglobulin (IgG). Dox promoted robust cardiomyocyte damage as measured by cTnT release in control co-cultures (FIG. 17A). These findings are qualitatively very similar to those obtained when r-cardiomyocytes were co-cultured with FGF2(WT)-MEFs (FIG. 15A), demonstrating that both mouse and human fibroblasts exert similar effects in co-culture with r-cardiomyocytes. Addition of Hi-Ab nearly eliminated Dox-induced damage (FIG. 17A), indicating that neutralization of paracrine human Hi-FGF2 was protective.

In a parallel experiment, r-cardiomyocytes were separated from h-Fib^{iPSC} by plating the latter in Transwell permeable inserts in the presence of Hi-Ab or control IgG; Dox-induced damage (cTnT) was reduced by 50% in the presence of Hi-Ab (FIG. 17B). In a third approach, r-cardiomyocytes were cultured with conditioned medium from h-Fib^{iPSC} in the presence of Hi-Ab or control IgG. Hi-Ab reduced Dox-induced damage (LDH release) significantly by 25% (FIG. 17C).

A different preparation of neutralizing antibodies (Neu-Ab) raised against the core Lo-FGF2 sequence present in both types of isoforms) is capable of recognizing, and neutralizing Lo-as well as Hi-FGF2, as demonstrated in our previous studies: Neu-Ab was capable of neutralizing the paracrine activity of Hi- or Lo-FGF2, individually overexpressed in r-cardiomyocytes (41); neu-Ab could also detect human Hi-as well as Lo-FGF-2 overexpressed in human embryonic kidney 293 cells (9). It is therefore expected that Neu-Ab is capable of neutralizing extracellular/paracrine Hi- and Lo-FGF2 in our co-cultures. R-cardiomyocytes were incubated with h-Fib^{iPSC} conditioned medium in the presence of Neu-Ab or control IgG. No protection was observed in the presence of Neu-Ab; in contrast to the protection seen with Hi-Ab, Neu-Ab elicited a small but significant increase in Dox-induced damage compared to control IgG (FIG. 17D).

Materials and Methods

Animals. Mice genetically engineered by Doetchman, T. and colleagues as to: express no FGF2, Fgf2tm1Doe, termed by us as FGF2(−), express only Hi-FGF2, Fgf2tm2Doe, termed by us as FGF2(Hi); express only Fgf2tm3Doe, termed by us as FGF2(Lo), and bred to C57BL/6J background, as well as wild type C57BL/6J mice (FGF2(WT)), were purchased from Jackson laboratories. Creation and characterization of these mouse models has been described in detail previously (11-13).

In vivo model for acute Doxorubicin effects. Young, 8 week old, male or female mice from the FGF2(WT), FGF2(−), FGF2(Hi), FGF2(Lo) groups were each randomly divided into saline-treated (n=10) and Dox-treated (n=10) groups. Dox groups received 20 mg/kg Dox intraperitoneally, while the saline groups were injected with an equal volume of normal saline. At day 10 post-Dox, mice were killed humanely and hearts collected for protein extraction and analysis by western blotting. Body weights were recorded at baseline and at day 10 post-Dox.

In vivo model for acute cardiotoxic effects. To induce acute cardiac injury a single intraperitoneal injection of the anthracycline Doxorubicin (Dox) was used at 20 mg/kg. This model is meant for proof-of principle studies of acute Dox cardiotoxicity and not intended to simulate the chronic effects of repeated Dox injections as would occur in anti-cancer treatments of different types of cancer patients. Eight-week-old groups of male or female FGF2(WT) and FGF2(Lo) mice were each randomly divided into saline- and Dox-treated groups; sample size was n=10 unless stated otherwise. At day 10 post-Dox treatment, mice were killed humanely by administering ketamine (150 mg/kg) and xylazine (10 mg/kg) intraperitoneally for induction of deep terminal anesthesia. Hearts were then collected for biochemical analyses.

Echocardiography. Echocardiography of conscious mice was done at baseline and daily for up to 10 days post-Dox, as previously described (29). Briefly, two-dimensional and tissue Doppler imaging was used to assess mouse cardiac function using a 13-MHz linear array ultrasound probe (Vivid 7, GE Medical Systems, Milwaukee, Wis., USA). Images were processed offline using EchoPAC v110.0.0 PC software. Left ventricular ejection fraction (EF) was calculated by manually tracing the endocardial contours in long-axis views. Fractional shortening (FS) and LVEDD was measured using the M-mode and short-axis views.

In vitro experiments. Neonatal rat ventricular cardiomyocytes (referred to as ‘cardiomyocytes’) were isolated from 1-2 day old rat pups as previously described (10).

Mouse embryonic fibroblasts (MEFs). Prepared as described (30).

MEF-Cardiomyocyte co-cultures. Cardiomyocytes were plated at a density of 5×10⁴ cells/cm² on collagen (Corning, #354236) coated glass coverslips in the presence of 20% fetal bovine serum (FBS) in Hams F-10 culture medium; 24 hours later the media was changed and MEFS from FGF2(WT), FGF2(−), FGF2(Hi), and FGF2(Lo) strains (2×10⁴/cm²) were added to the cultures in low-serum medium (0.5% FBS, 1% insulin, 1% transferrin/selenium, and 1% ascorbic acid) in Dulbecco's modified Eagle's medium (DMEM). After 48 hours, co-cultures were incubated with low serum medium containing 0.5 μM of Dox or saline. Conditioned media were collected 24 h later, and stored at −80° C. Cells attached on coverslips were used for the Calcein-AM/Cobalt chloride mPTP assay.

Primary mouse embryonic fibroblasts (MEFs)-r-cardiomyocyte co-cultures. MEFs were isolated as described (30). R-cardiomyocytes were plated at a density of 5×10⁴ cells/cm² on collagen (Corning, #354236) coated glass coverslips in the presence of 20% fetal bovine serum (FBS) in Hams F-10 culture medium; 24 hours later the media was changed and MEFS from FGF2(WT) and FGF2(Lo) strains (2×10⁴/cm²) were added to the cultures in low-serum medium (0.5% FBS, 1% insulin, 1% transferrin/selenium, and 1% ascorbic acid) in Dulbecco's modified Eagle's medium (DMEM). After 48 hours, co-cultures were incubated with low serum medium supplemented or not with Dox, 0.5 μM. Conditioned media were collected 24 h later, and stored at −80° C. Cells attached on coverslips were used for the Calcein-AM/Cobalt chloride mPTP assay.

Human induced pluripotent stem cells (iPSC)-derived fibroblasts. As described in our recent study, iPSC were derived from peripheral blood mononuclear cells (PBMNC) (46). All the protocols were approved by the Research Ethics Board, University of Manitoba. Briefly, blood samples were derived from healthy individuals, and PBMNC were isolated using the Lympholyte H kit (Cedarlane). PBMNC were reprogrammed to iPSCs using CytoTuneTM-iPS 2.0 Sendai Reprogramming Kit (ThermoFisher Scientific). The pluripotency of the established iPSC line (SGC 005.2) was checked through immunostaining with anti-Oct4, -Sox2, -Nanog, -Tra-1-60, -Tra-1-81 and -SSEA4 and tri-lineage differentiation. The iPSC were differentiated into fibroblasts (iPSC-Fibs) using a previously described protocol (32). The iPSC colonies were cultured in low attachment 6 well dishes (ThermoFisher Scientific) to form embryoid bodies (EBs). The EBs were then cultured in DMEM-High Glucose (ThermoFisher Scientific) supplemented with 10% FBS, 1× non-essential amino acid and 0.1 mM β-mercaptoethanol. The EBs were then plated onto 0.1% gelatin-coated dishes in the EB medium on day 8 at 1-2 EBs per 6-cm plate. After 12 days of culture, only the outgrowing cells were collected, trypsinized and plated onto non-gelatin coated dishes. These were passaged twice and then immunostained for fibroblast-specific markers (vimentin, fibroblast-surface protein and heat shock protein 47). Only those culture plates which showed >99% staining for all the three markers were used for further experiments.

Fib^{iPSC}-Cardiomyocyte co-cultures. Cardiomyocytes were plated at a density of 5×10⁴ cells/cm² on collagen coated plates in the presence of 20% (FBS) in Hams F-10 culture medium. After 24 hours, the media was changed to low serum medium (0.5% FBS, 1% insulin, 1% transferrin/selenium, 1% ascorbic acid, and 1% bovine serum albumin) in Dulbecco's modified Eagle's medium (DMEM). FibiPSC were added to the cultures at a density of 3×10⁴/cm². Human Hi-FGF2 antibody (Hi-Ab, 20 μg/ml) or non-immune rabbit IgG was added to the culture after 6 hours. The Hi-Ab and rabbit IgG were supplemented in the co-cultures every 24 hours. After 48 hours of co-culturing Cardiomyocytes with Fibs^{iPSC} the media was refreshed, Hi-Ab or rabbit IgG supplemented, and Dox (0.5 μM) or saline was added to the cultures for 24 hours. Subsequently, the media was collected and stored at −80° C.

Fib^{iPSC}-r-Cardiomyocyte Transwell co-cultures. After seeding cardiomyocytes 5×10⁴ cells/cm² on collagen coated plates in the presence of 20% (FBS) in Hams F-10 culture medium for 24 hours, the media was changed to low serum medium. Transwell permeable supports (1 μm pore size) were used to co-culture Fib^{iPSC} (5×10⁴ cells/cm²) with cardiomyocytes. The co-cultures were supplemented with 20 μg/ml Hi-Ab or rabbit IgG every 24 hours. After 48 hours Dox (0.5 μM) or saline was added to the cultures for 24 hours before the media was collected and stored at −80° C.

R-cardiomyocytes cultured with Fib^{iPSC} conditioned media. Fib^{iPSC} were cultured in 100 mm dishes in 10% FBS in DMEM until 80% confluent. The media was then changed to 5 ml of low serum medium (0.5% FBS, 1% insulin, 1% transferrin/selenium, 1% ascorbic acid, and 1% bovine serum albumin). Every 24 hours 2.5 ml was removed for experiments and replaced with fresh. Cardiomyocytes were seeded on collagen coated plates 5×10⁴ cells/cm² in the presence of 20% (FBS) in Hams F-10 culture medium for 24 hours. Then the media was replaced with 1 ml of fresh Fib^{iPSC} conditioned media supplemented with Hi-Ab (20 μg/ml) or rabbit IgG; in a different experiment, a monoclonal anti-total-FGF2 neutralizing antibody, or mouse immunoglobulin, were used at 25 ug/ml. Every 24 hours 1 ml of media containing the neutralizing antibody or unrelated immunoglobulin was added to the cultures. After 48 hours, Dox (0.5 μM) or saline was added to the cultures and, a day later, the media was collected and stored at −80° C. until further analysis.

Protein (western) immunoblotting. At the end of the in vivo study, hearts were collected, snap frozen in liquid nitrogen, and stored at −80° C. Hearts were powdered with liquid nitrogen using a mortar and pestle prior to being homogenized in 10 mM Tris-HCl pH 7.4, 100 mM NaCl, 300 mM sucrose, 2 mM MgCl₂, 1% thiodiglycol, 60 mM B glycerophosphate, 10 mM NaF and an equal volume of 20% glycerol, 100 mM Tris/HCl PH=6.8, and 2% sodium dodecyl sulphate (SDS). All buffers were supplemented (1:100) with protease inhibitor cocktail (Sigma-Aldrich, #8304) and phosphatase inhibitor cocktail set II and IV (Calbiochem, #524625 and #524628). Samples were boiled, sonicated, and centrifuged 15 minutes at 21,000 g to remove cellular debris. Protein concentration was measured by bicinchoninic acid assay. Following SDS-polyacrylamide gel electrophoresis and protein transfer to polyvinylidene fluoride (PVDF) membranes, total protein was stained using 2% Ponceau S (Sigma-Aldrich, P3504) (w/v) in 30% trichloroacetic acid to assess overall protein transfer. Non-specific binding sites were blocked by incubation in 10% milk/TBS-T for 1 hour at room temperature. The following antibodies were used: Bnip3L/Nix (Santa Cruz, 1:1000, sc-28240), and mouse monoclonal Bnip3 (1:1000, as previously described in (31)). Loading was normalized by Ponceau staining of the whole membrane and all the graphs represent values normalized to Ponceau staining.

Protein extraction and immunoblotting (western blot). At the end of the in vivo study, hearts were collected, snap frozen in liquid nitrogen, and stored at −80° C. For total protein extraction, hearts were powdered in liquid nitrogen using a mortar and pestle prior to being homogenized in 10 mM Tris-HCl pH 7.4, 100 mM NaCl, 300 mM sucrose, 2 mM MgCl₂, 1% thiodiglycol, 60 mM B glycerophosphate, 10 mM NaF and an equal volume of 20% glycerol, 100 mM Tris/HCl PH=6.8, and 2% sodium dodecyl sulphate (SDS). All buffers were supplemented (1:100) with protease inhibitor cocktail (Sigma-Aldrich, #8304) and phosphatase inhibitor cocktail set II and IV (Calbiochem, #524625 and #524628). Samples were boiled, sonicated, and centrifuged for 15 min at 21,000×g to remove residual debris. Protein concentration was measured using the bicinchoninic acid assay from Sigma. Following SDS-polyacrylamide gel electrophoresis and protein transfer to polyvinylidene fluoride (PVDF) membranes, total protein was stained using 2% Ponceau S (Sigma-Aldrich, P3504) (w/v) in 30% trichloroacetic acid to assess overall protein transfer. Non-specific binding sites were blocked by incubation in 10% milk/TBS-T for 1 hour at room temperature. The following antibodies were used: mouse monoclonal Bnip3 (1:1000), as previously described (41) and cTroponin-T (RV-C2, Developmental Studies Hybridoma Bank (DSHB), 1:200). The original densitometry values from the Western blots were adjusted based on corresponding Ponceau S staining of the whole lane.

Hi-FGF2 antibody. Anti-Hi-FGF2 antibody (Hi-Ab) against 12 amino acids from the N-terminal section of the Hi-FGF2 “GRGRAPERVG” (SEQ ID NO:1) was made in rabbit and affinity purified by Genscript. Characterization was done as we described previously, for a similar preparation anti-Hi-FGF2 (9). Affinity purified Hi-Ab was used at a concentration of 20 μg/ml in in vitro models.

Immunoprecipitation. A clarified (150 μg) protein solution, extracted from Fib^{iPSC} cultures directly into radioimmunoprecipitation, RIPA, buffer (150 mM NaCl, 1% (v/v) NP-40, 0.25% (w/v) deoxycholate, 0.1% (w/v) SDS, 50 mM Tris-HCl pH 8.0, 1 mM EGTA, 1 mM EDTA, 1 mM Na₃VO₄) supplemented with protease inhibitors, was mixed with 3 μg of purified Hi-Ab, followed by the addition of protein-A conjugated to Dynabeads™ (Thermofisher Scientific #10001D) to isolate antigen-antibody complexes, as per manufacturer's instructions. Immunoblotting was performed as described (9).

Mitochondrial permeability transition pore (mPTP) assay. Formation of mPTP was visualized as a reduction in the intensity of mitochondrial calcein staining using the Image-iT™ LIVE Mitochondrial Transition Pore Assay Kit (135103; Thermofisher), according to the manufacturer instructions. Image J software was used to measure individual cell fluorescence intensity of Calcein-AM (green). Mitochondria were counterstained (red) with the Mitotracker reagent in the kit.

Statistical analysis: For multiple comparisons between the groups two-way ANOVA with Tukey post hoc test and one-way ANOVa with Tukey post hoc test were used, as required. GraphPad Prism 6 software used to perform statistical analysis. The p<0.05 was considered as statistically significant. For the in vivo echocardiography experiments, data were obtained from 7-10 mice per group, with the exception of male FGF2(−) mice that due to high mortality had an n=3 at days 6-10 post-Dox. The scope of the claims should not be limited by the preferred embodiments set forth in the examples but should be given the broadest interpretation consistent with the description as a whole.

TABLE 1
Echocardiography and heart weight/body weight parameters

| | | |
|---|---|---|
| Mouse Strain | Saline | Dox |

FGF2(WT)

| | | |
|---|---|---|
| Male mice | | |
| LVESD, mm | 1.58 ± 0.06 | 1.95 ± 0.05*† |
| PWT, mm | 0.94 ± 0.07 | 0.97 ± 0.05 |
| HR, beats/min | 712 ± 39 | 665 ± 27 |
| Heart weight/body | 0.46 ± 0.03 | 0.44 ± 0.06 |
| weight, × 10⁻² | | |
| Female mice | | |
| LVESD, mm | 1.54 ± 0.04 | 1.83 ± 0.08*† |
| PWT, mm | 0.95 ± 0.06 | 0.89 ± 0.05 |
| HR, beats/min | 723 ± 34 | 645 ± 73 |
| Heart weight/body | 0.44 ± 0.03 | 0.43 ± 0.02 |
| weight, × 10⁻² | | |

FGF2(Lo)

| | | |
|---|---|---|
| Male mice | | |
| LVESD, mm | 1.51 ± 0.11 | 1.53 ± 0.07 |
| PWT, mm | 0.90 ± 0.09 | 0.93 ± 0.04 |
| HR, beats/min | 700 ± 41 | 658 ± 85 |
| Heart weight/body | 0.45 ± 0.03 | 0.43 ± 0.04 |
| weight × 10⁻² | | |
| Female mice | | |
| LVESD, mm | 1.41 ± 0.05 | 1.42 ± 0.08 |
| PWT, mm | 0.89 ± 0.06 | 0.84 ± 0.04 |
| HR, beats/min | 683 ± 30 | 670 ± 33 |
| Heart weight/body | 0.48 ± 0.05 | 0.44 ± 0.03 |
| weight × 10⁻² | | |

Data are means ± SD. Echocardiography and heart weight/body weight parameters from the wild-type fibroblast growth factor 2 [FGF2(WT)] and low-molecular-weight FGF2 [FGF2(Lo)] groups at day 10 post-doxorubicin (Dox) or postsaline administration are shown. Comparisons between groups were made using two-way ANOVA (variables are strain and treatment). LVESD, left ventricular end-systolic diameter; PWT, posterior wall thickness; HR, heart rate.
*Significant differences between saline- and Dox-treated groups within the same strain; †significant differences between FGF(WT) versus FGF(Lo) either after saline or after Dox treatment.

### REFERENCES

1. Levis B E, Binkley P F, and Shapiro C L. Cardiotoxic effects of anthracycline-based therapy: what is the evidence and what are the potential harms? The Lancet Oncology. 2017; 18(8):e445-e56.
2. Zamorano J L, Lancellotti P, Rodriguez Munoz D, Aboyans V, Asteggiano R, Galderisi M, et al. 2016 ESC Position Paper on cancer treatments and cardiovascular toxicity developed under the auspices of the ESC Committee for Practice Guidelines: The Task Force for cancer treatments and cardiovascular toxicity of the European Society of Cardiology (ESC). European heart journal. 2016; 37(36):2768-801.
3. Tebbi C K, London W B, Friedman D, Villaluna D, De Alarcon P A, Constine L S, et al. Dexrazoxane-associated risk for acute myeloid leukemia/myelodysplastic syndrome and other secondary malignancies in pediatric Hodgkin's disease. Journal of clinical oncology: official journal of the American Society of Clinical Oncology. 2007; 25(5):493-500.
4. Schloemer N J, Brickler M, Hoffmann R, Pan A, Simpson P, McFadden V, et al. Administration of Dexrazoxane Improves Cardiac Indices in Children and Young Adults With Acute Myeloid Leukemia (AML) While Maintaining Survival Outcomes. Journal of pediatric hematology/oncology. 2017; 39(5): e254-e8.
5. Hensley M L, Hagerty K L, Kewalramani T, Green D M, Meropol N J, Wasserman T H, et al. American Society of Clinical Oncology 2008 clinical practice guideline update: use of chemotherapy and radiation therapy protectants. Journal of clinical oncology: official journal of the American Society of Clinical Oncology. 2009; 27(1):127-45.
6. Kardami E, Detillieux K, Ma X, Jiang Z, Santiago J J, Jimenez S K, et al. Fibroblast growth factor-2 and cardioprotection. Heart failure reviews. 2007; 12(3-4):267-77.
7. Wang J, Nachtigal M W, Kardami E, and Cattini P A. FGF-2 protects cardiomyocytes from doxorubicin damage via protein kinase C-dependent effects on efflux transporters. Cardiovasc Res. 2013; 98(1):56-63.
8. Koleini N, Nickel B E, Wang J, Roveimiab Z, Fandrich R R, Kirshenbaum L A, et al. Fibroblast growth factor-2-mediated protection of cardiomyocytes from the toxic effects of doxorubicin requires the mTOR/Nrf-2/HO-1 pathway. Oncotarget. 2017; 8(50): 87415-30.
9. Santiago J J, McNaughton L J, Koleini N, Ma X, Bestvater B, Nickel B E, et al. High molecular weight fibroblast growth factor-2 in the human heart is a potential target for prevention of cardiac remodeling. PloS one. 2014; 9(5):e97281.
10. Santiago J J, Ma X, McNaughton L J, Nickel B E, Bestvater B P, Yu L, et al. Preferential accumulation and export of high molecular weight FGF-2 by rat cardiac non-myocytes. Cardiovasc Res. 2011; 89(1):139-47.
11. Zhou M, Sutliff R L, Paul R J, Lorenz J N, Hoying J B, Haudenschild C C, et al. Fibroblast growth factor 2 control of vascular tone. Nature medicine. 1998; 4(2):201-7.
12. Garmy-Susini B, Delmas E, Gourdy P, Zhou M, Bossard C, Bugler B, et al. Role of fibroblast growth factor-2 isoforms in the effect of estradiol on endothelial cell migration and proliferation. Circulation research. 2004; 94(10): 1301-9.
13. Azhar M, Yin M, Zhou M, Li H, Mustafa M, Nusayr E, et al. Gene targeted ablation of high molecular weight fibroblast growth factor-2. Developmental dynamics: an official publication of the American Association of Anatomists. 2009; 238(2):351-7.
14. O'Connell J L, Romano M M, Campos Pulici E C, Carvalho E E, de Souza F R, Tanaka D M, et al. Short-term and long-term models of doxorubicin-induced cardiomyopathy in rats: A comparison of functional and histopathological changes. Experimental and toxicologic pathology: official journal of the Gesellschaft fur Toxikologische Pathologie. 2017; 69(4):213-9.
15. Florkiewicz R Z, and Sommer A. Human basic fibroblast growth factor gene encodes four polypeptides: three initiate translation from non-AUG codons. Proceedings of the National Academy of Sciences of the United States of America. 1989; 86(11):3978-81.
16. Piotrowicz R S, Maher P A, and Levin E G. Dual activities of 22-24 kDA basic fibroblast growth factor: inhibition of migration and stimulation of proliferation. J Cell Physiol. 1999; 178(2):144-53.
17. Pellieux C, Foletti A, Peduto G, Aubert J F, Nussberger J, Beermann F, et al. Dilated cardiomyopathy and impaired cardiac hypertrophic response to angiotensin II in mice lacking FGF-2. J Clin Invest. 2001; 108(12):1843-51.
18. Virag J A, Rolle M L, Reece J, Hardouin S, Feigl E O, and Murry C E. Fibroblast growth factor-2 regulates myocardial infarct repair: effects on cell proliferation, scar contraction, and ventricular function. Am J Pathol. 2007; 171(5):1431-40.
19. Liu L, Pasumarthi K B, Padua R R, Massaeli H, Fandrich R R, Pierce G N, et al. Adult cardiomyocytes express functional high-affinity receptors for basic fibroblast growth factor. The American journal of physiology. 1995; 268(5 Pt 2):H1927-38.
20. Jiang Z S, Jeyaraman M, Wen G B, Fandrich R R, Dixon I M, Cattini P A, et al. High- but not low-molecular weight FGF-2 causes cardiac hypertrophy in vivo; possible involvement of cardiotrophin-1. Journal of molecular and cellular cardiology. 2007; 42(1):222-33.
21. Liao S, Bodmer J, Pietras D, Azhar M, Doetschman T, and Schultz Jel J. Biological functions of the low and high molecular weight protein isoforms of fibroblast growth factor-2 in cardiovascular development and disease. Developmental dynamics: an official publication of the American Association of Anatomists. 2009; 238(2):249-64.
22. Pintucci G, Quarto N, and Rifkin D B. Methylation of high molecular weight fibroblast growth factor-2 determines post-translational increases in molecular weight and affects its intracellular distribution. Mol Biol Cell. 1996; 7(8):1249-58.
23. Feige J J, Ling N, and Baird A. Phosphorylation of basic fibroblast growth factor by purified protein kinase C and the identification of a cryptic site of phosphorylation. Biochem Biophys Res Commun. 1991; 175(1):31-6.
24. Piotrowicz R S, Ding L, Maher P, and Levin E G. Inhibition of cell migration by 24-kDa fibroblast growth factor-2 is dependent upon the estrogen receptor. The Journal of biological chemistry. 2001; 276(6):3963-70.
25. Sheikh F, Sontag D P, Fandrich R R, Kardami E, and Cattini P A. Overexpression of FGF-2 increases cardiac myocyte viability after injury in isolated mouse hearts. American journal of physiology Heart and circulatory physiology. 2001; 280(3):H1039-50.
26. Zhang S, Liu X, Bawa-Khalfe T, Lu L S, Lyu Y L, Liu L F, et al. Identification of the molecular basis of doxorubicin-induced cardiotoxicity. Nature medicine. 2012; 18(11):1639-42.
27. Lebrecht D, Setzer B, Ketelsen U P, Haberstroh J, and Walker U A. Time-dependent and tissue-specific accumulation of mtDNA and respiratory chain defects in chronic doxorubicin cardiomyopathy. Circulation. 2003; 108(19):2423-9.
28. Dhingra R, Margulets V, Chowdhury S R, Thliveris J, Jassal D, Fernyhough P, et al. Bnip3 mediates doxorubicin-induced cardiac myocyte necrosis and mortality through changes in mitochondrial signaling. Proceedings of the National Academy of Sciences of the United States of America. 2014; 111(51):E5537-44.
29. Bordun K A, Premecz S, daSilva M, Mandal S, Goyal V, Glavinovic T, et al. The utility of cardiac biomarkers and echocardiography for the early detection of bevacizumab- and sunitinib-mediated cardiotoxicity. American journal of physiology Heart and circulatory physiology. 2015; 309(4):H692-701.
30. Mesaeli N, and Phillipson C. Impaired p53 expression, function, and nuclear localization in calreticulin-deficient cells. Mol Biol Cell. 2004; 15(4):1862-70.
31. Ray R, Chen G, Vande Velde C, Cizeau J, Park J H, Reed J C, et al. BNIP3 heterodimerizes with Bcl-2/Bcl-X(L) and induces cell death independent of a Bcl-2 homology 3 (BH3) domain at both mitochondrial and nonmitochondrial sites. The Journal of biological chemistry. 2000; 275(2):1439-48.
32. Chen H F, Chuang C Y, Shieh Y K, Chang H W, Ho H N, Kuo H C. Novel autogenic feeders derived from human embryonic stem cells (hESCs) support an undifferentiated status of hESCs in xeno-free culture condi-tions. Hum Reprod 24: 1114-1125, 2009. doi:10.1093/humrep/dep003.
33. Cheng Y, Li Z, Kardami E, Loh Y P. Neuroprotective effects of LMW and HMW FGF2 against amyloid beta toxicity in primary cultured hippocampal neurons. Neurosci Lett 632: 109-113, 2016. doi:10.1016/j.neulet.2016.08.031.
34. Feridooni T, Hotchkiss A, Remley-Carr S, Saga Y, Pasumarthi K B. Cardiomyocyte specific ablation of p53 is not sufficient to block doxoru-bicin induced cardiac fibrosis and associated cytoskeletal changes. PLoS One 6: e22801, 2011. doi:10.1371/journal.pone.0022801.
35. Dixon I M, Kardami E. Acute protection of ischemic heart by FGF-2: involvement of FGF-2 receptors and protein kinase C. Am J Physiol Heart Circ Physiol 282: H1071-H1080, 2002. doi:10.1152/ajpheart.00290.2001.
36. Jiang Z S, Srisakuldee W, Soulet F, Bouche G, Kardami E. Non-angiogenic FGF-2 protects the ischemic heart from injury, in the presence or absence of reperfusion. Cardiovasc Res 62: 154-166, 2004. doi:10.1016/j.cardiores.2004.01.009.
37. Jiang Z S, Wen G B, Tang Z H, Srisakuldee W, Fandrich R R, Kardami E. High molecular weight FGF-2 promotes postconditioning-like cardio-protection linked to activation of the protein kinase C isoforms Akt and p70 S6 kinase. Can J Physiol Pharmacol 87: 798-804, 2009. doi:10.1139/Y09-049.
38. Kole D, Grella A, Dolivo D, Shumaker L, Hermans W, Dominko T. High molecular weight FGF2 isoforms demonstrate canonical receptor-mediated activity and support human embryonic stem cell self-renewal. Stem Cell Res (Amst) 21: 106-116, 2017. doi:10.1016/j.scr.2017.04.006.
39. Koleini N, Kardami E. Autophagy and mitophagy in the context of doxorubicin-induced cardiotoxicity. Oncotarget 8: 46663-46680, 2017. doi:10.18632/oncotarget.16944.
40. Ma X, Dang X, Claus P, Hirst C, Fandrich R R, Jin Y, Grothe C, Kirshenbaum L A, Cattini P A, Kardami E. Chromatin compaction and cell death by high molecular weight FGF-2 depend on its nuclear local-ization, intracrine ERK activation, and engagement of mitochondria. J Cell Physiol 213: 690-698, 2007. doi:10.1002/jcp.21139.
41. Pasumarthi K B, Kardami E, Cattini P A. High and low molecular weight fibroblast growth factor-2 increase proliferation of neonatal rat cardiac myocytes but have differential effects on binucleation and nuclear morphology. Evidence for both paracrine and intracrine actions of fibro-blast growth factor-2. Circ Res 78: 126-136, 1996. doi:10.1161/01.RES. 78.1.126.
42. Srisakuldee W, Jeyaraman M M, Nickel B E, Tanguy S, Jiang Z S, Kardami E. Phosphorylation of Connexin-43 at serine 262 promotes a cardiac injury-resistant state. Cardiovasc Res 83: 672-681, 2009. doi:10.1093/cvr/cvp142.
43. Vengellur A, LaPres J J. The role of hypoxia inducible factor lalpha in cobalt chloride induced cell death in mouse embryonic fibroblasts. Toxicol Sci 82: 638-646, 2004. doi:10.1093/toxsci/kfh278.
44. Zhang J, Ye J, Altafaj A, Cardona M, Bahi N, Llovera M, Callas X, Cook S A, Comella J X, Sanchis D. EndoG links Bnip3-induced mito-chondrial damage and caspase-independent DNA fragmentation in isch-emic cardiomyocytes. PLoS One 6: e17998, 2011. doi:10.1371/journal.pone.0017998.
45. Zhang L, Parry G C, Levin E G. Inhibition of tumor cell migration by LD22-4, an N-terminal fragment of 24-kDa FGF2, is mediated by Neu-ropilin 1. Cancer Res 73: 3316-3325, 2013. doi:10.1158/0008-5472. CAN-12-3015.
46. Sequiera G L, Saravanan S, Dhingra S. Human-induced pluripotent stem cell-derived mesenchymal stem cells as an individual-specific and renew-able source of adult stem cells. Methods Mol Biol 1553: 183-190, 2017. doi:10.1007/978-1-4939-6756-8_14.

1. A method of reducing severity of cardiotoxicity caused by administration of an anthracycline to an individual comprising administering to said individual an effective amount of an FGF-2 modulating compound.

2. The method according to claim 1 wherein said FGF-2 modulating compound increases Lo-FGF-2 isoform levels relative to Hi-FGF-2 isoform levels.

3. The method according to claim 2 wherein Lo-FGF2 is administered to the individual in above-physiological concentrations.

4. The method according to claim 1 wherein said FGF-2 modulating compound decreases Hi-FGF-2 isoform levels relative to Lo-FGF-2 isoform levels.

5. The method according to claim 4 wherein the FGF-2 modulating compound is selected from the group consisting of: an antibody specific for Hi-FGF2; a micro-RNA, or si-RNA designed to selectively reduce Hi-FGF2 levels; an anti-sense oligonucleotide-based modulator; and a proteolytic-enzyme-based modulator which truncates Hi-FGF2 proteins to a version functionally similar to Lo-FGF2.

6. The method according to claim 1 wherein the FGF-2 modulating compound is selected from the group consisting of: exogenously added Lo-FGF-2; an antibody specific for Hi-FGF2; a micro-RNA, or si-RNA designed to selectively reduce Hi-FGF2 levels; an anti-sense oligonucleotide-based modulator; and a proteolytic-enzyme-based modulator which truncates Hi-FGF2 proteins to a version functionally similar to Lo-FGF2.

7. The method according to claim 1 wherein the FGF-2 modulating compound is selected from the group consisting of: an antibody specific for Hi-FGF2; a micro-RNA, or si-RNA designed to selectively reduce Hi-FGF2 levels; and an anti-sense oligonucleotide-based modulator;

8. The method according to claim 1 wherein the anthracycline is doxorubicin, epirubicin or daunorubicin.

9. The method according to claim 1 wherein the anthracycline is doxorubicin or epirubicin.

10. The method according to claim 1 wherein the individual is undergoing chemotherapy for cancer.

11. The method according to claim 1 wherein the FGF-2 modulator is a Hi-FGF-2-specific antibody.

12. The method according to claim 11 wherein the Hi-FGF-2-specific antibody is administered to the individual at least 1-3 hours before anthracycline administration.

13. A method of reducing severity of cardiotoxicity in an individual who had been administered an anthracycline comprising administering to said individual an effective amount of a FGF-2 modulating compound on a schedule.

14. The method according to claim 13 wherein the schedule is every 10 days to 1 month until cardiac function is monitored to be normal for 6 months.


SEQUENCE LISTING (1 sequence)

SEQ ID NO: 1 - protein, 10 aa, artificial
  Synthetic peptide corresponding to 12 N terminal amino acids of hFGF2
GRGRAPERVG